(19) United States
(12) Patent Application Publication
(10) Pub. No.: US 2022/0248946 A1
(43) Pub. Date: Aug. 11, 2022

(54) A SCOPE

(71) Applicant: National University of Ireland, Galway, Galway (IE)

(72) Inventors: Rory O'Callaghan, Galway (IE); Ivan Keogh, Galway (IE); Martin Gallagher, Galway (IE); Elizabeth McGloughlin, Galway (IE); Christina Walsh, Galway (IE); Martin O'halloran, Galway (IE); Jibran Ahmed Abbasi, Galway (IE); Barry Mcdermott, Galway (IE)

(21) Appl. No.: 17/629,224

(22) PCT Filed: Jul. 23, 2020
(86) PCT No.: PCT/EP2020/070872

§ 371 Date: Jan. 21, 2022

(30) Foreign Application Priority Data
Jul. 23, 2019 (EP) 19187833.9

(51) Int. Cl. A61B 1/227; A61B 1/00; A61B 1/05; A61B 1/06; A61B 1/12

(52) U.S. Cl. CPC A61B 1/227; A61B 1/00096; A61B 1/00183; A61B 1/121; A61B 1/00114; A61B 1/05; A61B 1/0627; A61B 1/00188

(57) ABSTRACT

A scope (1) for examining or surgically treating ears comprising a probe (2); at least one visualiser (3) on the probe having an optical configuration (7), and a light source (42) wherein the visualiser (3) is articulatable about a visualiser articulation axis (3a) by an orienting mechanism (4) for optimal viewing of the ear.

### RELATED APPLICATIONS

This application claims priority from EP19187833, the contents of which are hereby incorporated by reference.

### INTRODUCTION

This invention relates to a scope and more particularly to a medical scope such as an otoscope or an endoscope for examining and surgically treating ears.

### BACKGROUND OF THE INVENTION

Medical devices of various types are employed to examine and perform surgery on ears. For example, endoscopes and otoscopes (hereinafter referred to collectively as scopes) are instruments which are held against the eye to view and magnify the subject. These instruments have evolved with the addition of a camera so that the subject is viewed using a computer monitor or a video display.

Recurrent acute otitis media, otitis media with effusion, chronic secretory/suppurative otitis media continues to impact quality of life for millions of patients world-wide. Hearing loss, complications of inflammation and complications of treatment are daily challenges and, as a result, patients with these conditions (children and adults) frequently require invasive ear surgery. As ears are wrapped in dense bone, surgical access and visualization are essentials for safe ear surgery.

In traditional ear surgery, microscopic visualization is employed with a dynamic bi-manual surgical technique. However, a major disadvantage of this approach is the narrow field of view looking down into the ear canal which can lead to poorer visualization of disease in difficult to access areas in the middle ear, such as the sinus tympani and facial recess.

In order to improve the field of view, it is often necessary to expose the middle ear and attic area by performing a mastoidectomy. However, mastoidectomy procedures are associated with increased operating times, more serious complications, longer hospital stays and protracted recovery for patients.

In addition, trans canal surgery is generally performed through a speculum by looking through the microscope and using specialized instruments resulting in a narrow field of view.

Moreover, due to the large size of the microscopic equipment employed in ear surgeries, negative ergonomic issues arise—the surgeon is forced into an extended position due to the size of the operating microscope which can compromise the dexterity required of the surgeon during surgical procedures.

In addition, when using a traditional analogue otoscopes or endoscopes, the human eye is effectively the image sensor utilizing the magnification provided by the otoscope/endoscope optics. Accordingly, when the otoscope/endoscope is reoriented or rotated in the ear canal the image observed remains oriented with respect to the user (independent of the otoscope/endoscope). However, with a video otoscope or endoscope the image sensor is located on the scope. Accordingly, when the scope is reoriented or rotated (perhaps for the comfort of the patient) the image observed also reorients or rotates which can result in disorientation of the user.

Moreover, in use, tissue can frequently adhere to scopes which can obscure vision with the result that the scope must regularly be removed from a subject's ear during surgery for cleaning which interrupts and prolongs the surgical procedure.

Overall, difficulties are encountered by surgeons visualising and focusing on the internal structures of the ear during surgery using known scopes in which the ability to accurately focus the scope and orient the scope for optimal viewing is absent.

Moreover these same problems occur in all types of surgery in all areas of the body, the scope can also be used visualise interior structures of the body in any part of the body, for example such as those seen in surgeries and procedures such as laparoscopy, Ureteroscopy arthroscopy, FESS Sinus Surgery, Neurosurgery and spine surgery and Gastro Intestinal Surgery.

An object of the invention is to overcome at least some of the problems of the prior art.

### SUMMARY OF THE INVENTION

According to a first aspect, there is provided a scope for examining an internal part of the ear, the scope comprising: a probe formed by an elongate probe body for insertion into the internal ear structure; and a camera comprising an image sensor located within a housing; wherein the housing is rotatably coupled to a distal end of the probe body whereby the camera is rotatable relative to the probe body.

The housing forms part of the camera so that rotation of the housing causes a corresponding rotation of the camera. Any reference herein to rotation of the housing can be interpreted as providing corresponding rotation of the camera.

An outer surface of the rotatable camera may form an outer exposed surface of the scope. The outer surface is exposed during use.

The image sensor may be arranged to visualise through the outer surface of the rotatable camera which forms the outer exposed surface of the scope. The outer surface of the rotatable camera through which the image sensor may be arranged to visualise may be spaced apart from the probe body to maintain clearance between them and allow relative movement between them, wherein the camera is preferably arranged to rotate 360 degrees about its rotational axis

At least a portion of the camera may be disposed distally beyond the distal end of the probe body.

The probe body may comprise one or more tabs supporting the housing extending in a distal direction from the distal end of the probe body.

The one or more tabs may comprise a pair of tabs. The pair of tabs may be opposed to each other about a central longitudinal axis of the probe body. An axis of rotation about which the housing can rotate may extend between the tabs.

At least one distally-opening notch may be defined between the pair of tabs. The at least one notch may form a cut-out portion of the distal end of the probe body relative to the tabs. This allows the range of view of the camera to be increased. Specifically, the notches may be arranged to allow the camera to have an unblocked field of view having a viewing angle pointing in a direction having a proximal component (e.g. greater than perpendicular to a longitudinal axis of the probe, in a direction back towards the proximal end of the probe).

A pair of notches may be defined on opposing sides of the probe body between the pair of tabs, the notches being mutually aligned on an axis that is substantially perpendicularly to the axis of rotation.

A portion of the camera may extend distally beyond the portion of the probe body formed by the notch or notches.

The one or more tabs may comprise a single tab, the single tab being arranged to support the camera in a cantilever arrangement along the axis of rotation of the camera.

The housing may be rotatably coupled to the one or more tabs via respective one or more axis pins extending along an axis of rotation of the housing. e.g. each tab has a corresponding axis pin.

Each of the one or more axis pins may originate in the camera and terminate in a receptacle on the respective tab. Alternatively each of the axis pins originate in the respective tab and terminate in a receptacle in the camera housing. The arrangement of pins and receptacles may be reversed so that each may be provided on the camera or probe.

Each axis pin may comprise an electrical contact electrically coupled to the image sensor within the housing for delivering power to the image sensor.

The image sensor may be configured to generate image data and wherein the generated image data is transmitted via at least one of the axis pins. Electrical power and image data may be transmitted by the same one of the axis pins.

The probe may be configured to wirelessly transmit power to the camera. The probe may comprise one or more near field wireless power transmission components (e.g. one or more induction coils), and the camera comprises one or more corresponding near field receiving components (e.g. one or more induction coils), arranged to transfer power between each other.

One of the one or more near field power transmission/receiving components may be located in at least one of the one or more axis pins, and at least one corresponding wireless power transmission/receiving component is located in the respective receptacle for the axis pin or pins. e.g. in the camera or the respective tab. This arrangement is to prevent the possible contact of fluid with components which could carry electrical power.

The near field wireless power transmission/receiving components are further arranged to transmit data between the probe and the camera in addition to electrical power.

The scope may further comprise a data cable coupled to the camera.

The image sensor may be configured to generate image data. The generated image data may be transmitted via the cable.

The probe body may comprise an inner channel.

The inner channel may form an open mouth at its distal end for holding the camera, wherein the housing is partially received within the open mouth.

The scope may further comprise one or more self-cleaning modules. At least one of the self-cleaning modules may comprises a cleaning element, wherein the camera is rotatable relative to the cleaning element. This allows the cleaning element to clean the outer surface of the camera that is exposed during used.

The cleaning element may be positioned proximally relative to the camera. The cleaning element may have another suitable position relative to the camera including being positioned distally relative to the camera.

The cleaning element of the or each cleaning module may be fixed in position by attachment to the probe, and is arranged to engage with the rotatable camera at any point along the camera circumference as defined by its rotation axis.

At least one of the self-cleaning modules may comprise a cleaning element that is movable relative to the probe, the movable cleaning element preferably being a rotational element such as a brush, and wherein the movable cleaning element is optionally rotated independently or in communication with a camera orienting mechanism.

The cleaning element may be disposed within the inner channel of the probe body.

The cleaning element may be offset laterally with respect to the camera (e.g. offset in the direction perpendicular to the longitudinal axis of the probe body).

The cleaning element may be disposed between the pair of tabs.

The camera may be fitted with an optical configuration comprising a lens and wherein the image sensor is configured to generate image data from light received through the lens.

The optical configuration may further comprises a window forming part of the housing, the window aligned with the image sensor and the lens such that an optical path is defined between the image sensor and the window, the optical path extending through the lens.

A fluid filled gap may be defined between an inner surface of the window and an outer surface of the lens. The fluid filled gap may be an air filled gap.

The optical configuration may further comprise a focusing mechanism for focussing the lens. The focusing mechanism may provide relative movement between the lens and image sensor.

The focusing mechanism may comprise a memory metal component configured to focus the lens.

The focusing mechanism is electromechanically operated, and preferably comprises a microelectromechanical system.

The focusing mechanism may comprise a focusing lens, and may be arranged to vibrate the focusing lens or the image sensor to provide relative movement between them so that the distance between the focusing lens and the image sensor varies. The scope, or a data processing system coupled to the scope, may be arranged to sample the resulting image data to generate focused images. The image data may be sampled by selecting frames corresponding to a desired focal point along the range of motion of the lens.

The vibratable focusing lens/image sensor may be used to provide cleaning of an outer surface of the camera (e.g. the lens or window). The focusing mechanism may be selectively mechanically coupled to the outer surface of the camera by a switchable damper. The damper may be switchable between a damping condition in which vibration of the focusing mechanism is damped, and a locked condition in which vibration of the focusing mechanism is transmitted to the outer surface of the camera.

The focusing mechanism may be arranged to vibrate the focusing lens/image sensor at a frequency that is approximately the same as the resonant frequency of the outer lens or window of the camera. The focusing mechanism may be arranged to switch the frequency of vibration of the focusing lens and/or image sensor between a first frequency which is different from the resonant frequency of the lens or window of the camera and a second frequency that is approximately the same as the resonant frequency of the outer lens or window.

The vibration of the focusing lens/image sensor may be arranged to generate a cleaning air flow across the lens or window of the camera. The camera may comprise one or more vents. The vents may be arranged to provide a stream of air generated by pressure changes within the camera resulting from the vibration of the focusing lens/image sensor. The vents may be arranged adjacent the lens or window forming the outer surface of the camera. The vents may be formed by through holes extending through the lens or window forming the outer surface of the camera.

The camera may comprise a plurality of movable cleaning members in the form of cilia extending from the outer surface of the camera. For example, from an outer surface of the lens or window through which light is transmitted into the camera.

The cilia may be arranged to move relative to the outer surface of the camera to move debris across its surface away from the field of field of the image sensor.

The probe may comprise a plurality of movable cleaning members arranged on a surface of the probe body adjacent the camera. The movable cleaning members may be arranged to move relative to the probe body to move debris across the surface of the probe body. The plurality of movable cleaning members may be in the form of cilia. The probe may further comprise a suction system arranged to remove material collected by the cilia.

The probe body or camera may further comprise a cleaning fluid supply device arranged to provide a source of cleaning fluid to the movable cleaning members.

The camera may comprise a communication module configured to transmit and/or receive image data generated by the image sensor.

The communication module may be configured to transmit and/or receive image data at a frequency equal to or in excess of 2.4 GHz. The camera may be arranged to send raw image data from the image sensor wirelessly to the probe. The camera may comprise a processor and an RF unit in communication with an antenna. In this embodiment, the camera may not have an image processing electronics, thereby having minimal electronics for transmission of the image data from the probe to the camera. This allows the size of the camera to be minimised. In other embodiments, image processing may be provided in the camera.

The communication module on the probe may comprise a video processing unit to process the raw image data. The video processing unit may be arranged to compress the raw image data, preferably using an algorithm which is transmittable over a long distance or interpreted by a readily available software.

The camera communication module may be configured for wirelessly transmitting or receiving data to or from a communication module provided in the probe. The communication module provided in the probe may be less than 30 mm from the camera.

The camera wireless communication module may comprise an antenna system comprising an omnidirectional antenna (e.g. a monopole antenna) antenna and the probe communication module may comprise an antenna system comprising a single directional antenna.

The camera wireless communication module may comprise an antenna system comprising a monopole antenna and the probe communication module may comprise an antenna system comprising an omnidirectional antenna (e.g. a monopole antenna).

The camera wireless communication module may comprise an antenna system comprising two or more directional antenna having different orientations and the probe communication module may comprise an antenna system comprising a single directional antenna. The two or more single directional antenna provided in the camera may be selectively operated depending on the orientation of the camera relative to the probe. The two or more single directional antenna provided in the camera may be selectively operated depending on the orientation of the camera relative to the probe. The camera may be arranged to select the most advantageous antenna (e.g. the closest) to transmit the data to the probe using directional data sensed from within the camera. The camera may be arranged to switch off the other antennas apart from the most advantageous antenna.

The camera may be configured to transmit raw (i.e. unprocessed) image data from the camera to the probe via the wireless communication modules. This means that little or no image process takes place within the camera, and allows the camera to be made smaller.

The transmission distance between the antenna system of the probe and the antenna system of the camera may be less than 25 mm, preferably between 5-10 mm.

The scope may further comprise a light source incorporated into the camera.

The light source may be movable with the image sensor with respect to the probe body.

The probe may further comprise a light source for illuminating a field of view of the image sensor.

The scope may comprise an orientating mechanism configured to control the rotational movement of the housing relative to the probe body.

The orientating mechanism may comprise a belt orienting mechanism. The belt orienting mechanism may comprising a drive belt coupled to a drive shaft positioned at a proximal end of the probe and a driven shaft acting on the housing.

The drive belt may be wrapped around the drive shaft and/or the driven shaft at least twice.

The drive shaft may be operatively coupled to a stepper motor for rotating the drive shaft.

The orientating mechanism may comprise at least one cable coupled to two attachment points on a distal side of the housing, the attachment points being mutually opposed about an axis of rotation of the housing.

The orienting mechanism may comprise a first cable arranged to provide rotation of the camera in a first direction and a second cable arranged to provide rotation in a second direction about the rotational axis. The first and second cables may be connected to or within the camera (so that they extend from a point on the outer surface of the camera) and extend in opposite directions about the rotational axis of the camera around the housing of the camera.

The at least one cable or first and second cables may enter the camera at the same point on its circumference as defined by the rotation axis, preferably in different planes (e.g. normal to the rotational axis) relative to each other.

The cable or cables may be configured to provide rotation in excess of 175 degrees in either direction around the rotational axis of the camera from a centre position in which the image sensor points in a distal direction along a longitudinal axis of the probe.

The first cable may extend around a first side of the camera and the second cable extends around a second side of the camera, wherein the first cable extends from a channel in the probe body that is located on the side of the probe body corresponding to the second side of the camera, and wherein the second cable extends from a channel in the probe body that is located on the side of the probe body corresponding to the first side of the camera.

The first and second cables may extend from the same point on the camera (i.e. the same point around the circumference of the camera relative to the rotational axis.). The first and second cables may extend from the distal side of the camera (e.g. they may extend from the most distal point of the camera/housing when it is in an un-rotated, zero degree, position with the image sensor point in the distal direction along the longitudinal axis of the probe body).

The first cable may extend from a point on the camera on a first side relative to the rotational axis and extend around the camera around an opposing second side of the camera. The second cable may extend from a point on the second side and extend around the first side in the opposite direction. This may allow 360 degree rotation of the camera about the rotational axis.

The at least one cable or cables (first and second cables) in the preceding statements may be electrically connected to the image sensor within the housing so as to deliver electrical power to the image sensor.

The at least one cable or cables (first and second cables) in the preceding statements may be connected to the image sensor so as to transmit image data generated by the image sensor. They may carry both electrical power and data.

This may allow the orienting mechanism to provide both actuation of the camera and data/power transmission without the need for other connections/wireless communication.

The camera may have a uniform profile about its rotational axis. The camera may be substantially circular in section through its axis of rotation. It may in other embodiments be cylindrical. The camera may be generally spherical. The camera may have a circumference that is less than 6.5 mm measured through the rotational axis. The camera may have a circumference that is less than 3.5 mm measured through the rotational axis.

The probe may be configured to wirelessly transmit power to the visualizer. The probe may comprise one or more induction coils and the camera may comprise one or more induction coils arranged to transfer power between each other. The induction coils may be arranged to transfer energy by inductive coupling. The one or more induction coils provided at the probe may be arranged in the tabs supporting the camera. The one or more induction coils provided at the camera may be arranged in the axis pins of the camera.

The scope may further comprise a speculum for insertion into the patient's ear and wherein the probe body is moveable relative to the speculum.

According to a second aspect, there is provided a method of examining the internal ear structure of a patient, the method comprising: inserting a probe into the internal ear structure, the probe formed by an elongate probe body supporting a camera comprising an image sensor disposed within a housing, the housing being rotatably coupled to a distal end of the probe body whereby the camera is rotatable relative to the probe body; rotating the housing relative to the probe body to cause rotational movement of the image sensor relative to the internal ear structure; and wherein rotating the housing comprises rotating the housing through at least 90° relative to the probe body whilst viewing the internal ear structure throughout the rotational movement.

An outer surface of the rotatable camera may form an outer exposed surface of the scope. The outer surface is exposed during use.

The method may comprise rotating the camera from a first position in which the image sensor faces a distal direction to a second position in which the image sensor faces a proximal direction.

The method may further comprise rotating the camera (and housing) back to the first position.

Rotating the camera from the first position, to the second position and back to the first position may comprise rotating the camera (and housing) 360°.

Rotating the camera may comprise rotating the camera over a range in excess of 175 degrees in either direction around the rotational axis of the camera from a centre position in which the image sensor points in a distal direction along a longitudinal axis of the probe.

Rotating the camera may comprise rotating the camera using an orienting mechanism comprising a first cable arranged to provide rotation of the camera in a first direction and a second cable arranged to provide rotation in a second direction about the rotational axis, wherein preferably the first and second cables are connected to or within the camera and extend in opposite directions about the rotational axis of the camera around the housing of the camera.

The first cable may extend around a first side of the camera and the second cable extends around a second side of the camera, wherein the first cable extends from a channel in the probe body that is located on the side of the probe body corresponding to the second side of the camera, and wherein the second cable extends from a channel in the probe body that is located on the side of the probe body corresponding to the first side of the camera.

The method may comprise cleaning an outer surface of the camera.

Cleaning of the outer surface of the camera may comprise rotating the camera (and housing) relative to a cleaning element.

Rotating the camera (and housing) relative to the cleaning element may comprise wiping an outer surface of the camera with the cleaning element.

The camera may comprise a lens aligned with the optical sensor and wherein the method comprises focusing the lens.

Rotating the camera may comprise rotating the housing through at least 120° relative to the probe body.

According to a third aspect, there is provided a method of cleaning a scope for examining an internal part of the ear, wherein the scope comprises an image sensor disposed within a rotatable housing and wherein the scope further comprises a cleaning element, the method comprising: rotating the housing relative to the cleaning element; and using the cleaning element to clean an outer surface of the housing.

Cleaning the outer surface of the housing may comprise wiping the housing with the cleaning element by rotating the housing relative to the cleaning element.

Rotating the housing may comprises rotating the housing from a first position in which the optical sensor faces in a distal direction to a second position in which the optical sensor faces in a proximal direction.

The method may further comprise returning the housing to the first position.

Rotating the housing may comprises turning the housing through 360° about an axis of rotation of the housing transverse to a central longitudinal axis of the probe body.

Rotating the housing may comprise rotating the housing from a first position in which the optical sensor faces in a distal direction to a second position in which the image sensor faces generally perpendicularly relative to a central longitudinal axis of the probe body.

According to a fourth aspect, there is provided a camera for examining the internal ear structure of a patient, the camera comprising: a rod for insertion into the internal ear structure; and an optical sensor located within a housing; wherein the housing is rotatably coupled to a distal end of the rod and wherein at least a portion of the housing is disposed distally beyond the distal end of the rod.

The rod may comprise a pair of tabs supporting the housing extending in a distal direction from the distal end of the rod.

The tabs may be opposed to each other about a central longitudinal axis of the rod and wherein an axis of rotation about which the housing can rotate extends between the tabs.

At least one distally-opening notch may be defined between the pair of supporting tabs.

A pair of notches may be defined on opposing sides of the rod between the pair of tabs, the notches being mutually aligned on an axis that is substantially perpendicularly to the axis of rotation.

A portion of the housing may extend distally beyond a distal end of the tabs.

The housing may be rotatably coupled to the tabs via pins extending along an axis of rotation of the housing.

Each pin may comprise an electrical contact electrically coupled to the optical sensor within the housing for delivering power to the optical sensor.

The optical sensor may be configured to generate image data and wherein the generated image data is transmitted via at least one of the pins.

The camera may further comprise a data cable coupled to the housing.

The optical sensor may be configured to generate image data and wherein the generated image data is transmitted via the cable.

The rod may comprise an inner lumen.

The housing may be partially received within the inner lumen.

The camera may further comprise a cleaning element, wherein the housing is rotatable relative to the cleaning element.

The cleaning element may be positioned on a proximal side of the housing.

The cleaning element may be disposed within the inner lumen of the rod.

The cleaning element may be offset laterally with respect to the housing.

The cleaning element may be disposed between the pair of tabs.

The housing may comprise a lens and wherein the optical sensor is configured to generate image data from light received through the lens.

The housing may further comprise a window aligned with the optical sensor and the lens such that an optical path is defined between the optical sensor and the window that extends through the lens.

An air gap may be defined between an inner surface of the window and an outer surface of the lens.

The housing may comprise a focusing mechanism for focussing the lens.

The focusing mechanism may comprise a memory metal component configured to focus the lens.

The focusing mechanism may comprise a microelectromechanical system.

The housing may comprise a communication module configured to transmit and/or receive image data generated by the optical sensor.

The communication module may be configured to transmit and/or receive image data at a frequency in excess or equal to 2.4 GHz.

The housing may further comprise a light source for illuminating a field of view of the optical sensor.

The light source may be movable with the optical sensor with respect to the rod.

The rod further may comprise a light source for illuminating a field of view of the optical sensor.

The camera may comprise an orientating mechanism configured to control the rotational movement of the housing relative to the rod.

The orientating mechanism may comprise a drive belt coupled to a drive shaft positioned at a proximal end of the rod and a driven shaft acting on the housing.

The drive belt may be wrapped around the drive shaft and the driven shaft at least twice.

The drive shaft may be operatively coupled to a stepper motor for rotating the drive shaft.

The orientating mechanism may comprise at least one cable coupled to two attachment points on a distal side of the housing, the attachment points being mutually opposed about an axis of rotation of the housing.

The at least one cable may be electrically connected to the optical sensor within the housing so as to deliver electrical power to the optical sensor.

The at least one cable may be connected to the optical sensor so as to transmit image data generated by the optical sensor.

The housing may be substantially circular in section through an axis of rotation.

The housing may be generally spherical.

According to a fifth aspect, there is provided a scope comprising a camera according to the fourth aspect or any of the statements above.

The scope may further comprise a speculum for insertion into the patient's ear and wherein the rod is moveable relative to the speculum.

According to another aspect there is provided a method of examining the internal ear structure of a patient, the method comprising: inserting a rod into the internal ear structure, the rod supporting an optical sensor disposed within a rotatable housing; rotating the housing relative to the rod to cause rotational movement of the optical sensor relative to the internal ear structure; and wherein rotating the housing comprises rotating the housing through at least 90° relative to the rod whilst viewing the internal ear structure throughout the rotational movement.

The method may comprise rotating the housing from a first position in which the optical sensor faces a distal direction to a second position in which the optical sensor faces a proximal direction.

The method may further comprise rotating the housing back to the first position.

Rotating the housing from the first position, to the second position and back to the first position may comprise rotating the housing 360°.

The method may comprise cleaning an outer surface of the housing.

Cleaning the outer surface of the housing may comprise rotating the housing relative to a cleaning element.

Rotating the housing relative to the cleaning element may comprise wiping an outer surface of the housing with the cleaning element.

The housing may comprise a lens aligned with the optical sensor and wherein the method comprises focusing the lens.

Rotating the housing may comprise rotating the housing through at least 120° relative to the rod.

According to a seventh aspect, there is provided a method of cleaning a camera for examining the internal ear structure of a patient, wherein the camera comprises an optical sensor disposed within a rotatable housing and wherein the camera further comprises a cleaning element, the method comprising: rotating the housing relative to the cleaning element; and using the cleaning element to clean an outer surface of the housing.

Cleaning the outer surface of the housing may comprise wiping the housing with the cleaning element by rotating the housing relative to the cleaning element.

Rotating the housing may comprise rotating the housing from a first position in which the optical sensor faces in a distal direction to a second position in which the optical sensor faces in a proximal direction.

The method may further comprise returning the housing to the first position.

Rotating the housing may comprise turning the housing through 360° about an axis of rotation of the housing transverse to a central longitudinal axis of the rod.

Rotating the housing may comprise rotating the housing from a first position in which the optical sensor faces in a distal direction to a second position in which the optical sensor faces generally perpendicularly relative to a central longitudinal axis of the rod.

Any of the features of the fourth to seventh aspects given above may apply to the first to third aspects.

According to another aspect of the invention there is provided a scope for examining or surgically treating a part of the body comprising:
a probe;
at least one visualiser on the probe having an optical configuration, and
a light source,

wherein the visualiser is articulatable relative to the probe by an orienting mechanism for optimal viewing of the body part.

In one embodiment, the part of the body is a difficult to access part of the body, such as the internal part of the ear, nose, throat, or another body orifice.

In one embodiment, the orienting mechanism is configured to tilt or rotate the visualiser relative to the probe.

In one embodiment, the visualiser is articulatable about a visualiser articulation axis by an orienting mechanism for optimal viewing of the ear.

Preferably, the visualiser is rotatable about the visualiser articulation axis. More preferably, the visualiser has a uniform profile about its articulation axis.

Most preferably, the visualiser is substantially spherical. In one embodiment, the probe comprises a socket configured for receipt of the substantially spherical visualiser for rotation of the visualiser about multiple axes. In one embodiment, the orienting mechanism comprises a roller in contact with the substantially spherical visualiser, whereby rotation of the roller effects rotation of the substantially spherical visualiser. In one embodiment, the orientating mechanism comprises a first roller in contact with the substantially spherical visualiser and configured for rotation about a first axis of rotation, and a second roller in contact with the substantially spherical visualiser and configured for rotation about a second axis of rotation, wherein the first and second axes of rotation are optionally orthogonal to each other.

Suitably, the visualiser comprises an image sensor or a camera.

In one embodiment, the probe comprises a hinge.

Suitably, the orienting mechanism is a belt- or band-driven orienting mechanism, or a fluidic (i.e. pneumatic or hydraulic) orienting mechanism In one embodiment, the orienting mechanism comprises a wheel or cog operatively coupled to the visualiser. In one embodiment, the orienting mechanism comprises a magnetic force generator configured to act on the visualiser. In one embodiment, the scope is configured for manual actuation of the orienting mechanism. In one embodiment, the orienting mechanism comprises a motor configured to drive the articulation of the visualiser.

In a preferred embodiment, the scope further comprises a self-cleaning module for cleaning detritus from the visualiser. Preferably, the self-cleaning module comprises a cleaning blade and/or a soft cleaning material. In one embodiment, the visualiser is configured for movement relative to the self-cleaning module to clean the visualiser. In one embodiment, the self-cleaning module comprises a sheath configured for movement from a non-deployed position to a deployed position in which the sheath closes over the lens of the visualiser wiping the lens. In this embodiment, the visualiser may be configured for movement to a cleaning position when the sheath is deployed, for example retracted into the sheath and/or tilted to one side.

Preferably, the optical configuration comprises lens elements configured to allow for variable focusing and refocusing.

Suitably, the lens elements comprise an internal lens within the visualiser.

In a preferred embodiment, the lens elements comprise an external curved lens on the visualiser.

The invention also extends to a scope further comprising a stabilizer for supporting the scope. Preferably, the stabilizer comprises a speculum.

In a particularly preferred embodiment of the invention, the scope comprises an endoscope or an otoscope.

In another embodiment, the invention relates to a scope for examining or surgically treating a part of the body (for example a difficult to access part of the body such as the ear, nose or throat) comprising:
a probe;
at least one visualiser on the probe,
an optical configuration including image sensors, and
a light source

wherein the optical configuration is stacked in the probe to save space and reduce the profile of the probe. Preferably, the image sensors comprise image sensor boards.

Suitably, the optical configuration comprises first and second stacked fixed image sensors. Preferably, the optical configuration comprises corresponding first and second lens elements directing light to the image sensors. Advantageously, the optical configuration comprises a slidably adjustable front lens.

In one embodiment, the optical configuration comprises aspherical lenses to bypass the first fixed image sensor. Preferably the aspherical lenses comprise crescent profile lenses.

In a further embodiment, the invention relates to a scope for examining or surgically treating a part of the body (for example a difficult to access part of the body such as the ear, nose or throat) comprising:
a probe;
at least one visualiser on the probe having an optical configuration, and
a light source

wherein the visualiser is articulatable about a visualiser articulation axis by an orienting mechanism for optimal viewing of the ear and the articulatable visualiser is a tiltable camera

Preferably, the orienting mechanism comprises a directional lever.

Alternatively, the orienting mechanism comprises a directional wheel and the probe is slidable to adjust the depth of the probe. In a still further embodiment, the orienting mechanism comprises a slider either alone or in combination with the directional wheel and/or directional lever.

Preferably, the scope further comprises a stabilizer for supporting the tiltable visualiser in or on the stabilizer, the stabilizer being integral with the tiltable visualiser and being configured to stabilize the scope in an ear canal. More preferably, the probe is mountable on the stabilizer at a probe mounting.

Suitably, the stabilizer comprises a speculum holder. In a preferred embodiment, the stabilizer comprises a detachable or integrated speculum. Advantageously, light from the light source is transmissible through the speculum.

In a still further embodiment, the invention relates to a scope for examining or surgically treating a part of the body (for example a difficult to access part of the body such as the ear, nose or throat) comprising:
a probe;
at least one visualiser on the probe,
an optical configuration, and
a light source

wherein the optical configuration includes an angled prism adjacent the visualiser to assist in unobtrusive visualisation. Preferably, the visualiser comprises a camera.

In a further embodiment, the invention relates to a scope for examining or surgically treating a part of the body (for example a difficult to access part of the body such as the ear, nose or throat) comprising:
a probe;
at least one visualiser on the probe having an optical configuration, and
a light source

wherein the visualiser is articulatable about a visualiser articulation axis by an orienting mechanism for optimal viewing of the part of the body, the articulatable visualiser is a tiltable camera and the orienting mechanism comprises a living hinge on the probe. Preferably, the living hinge comprises an external living hinge. Alternatively, the living hinge comprises an internal living hinge. Suitably, the living hinge comprises a double living hinge Advantageously, the double living hinge comprises a spring-operated double living hinge.

Optionally, the optical configuration includes an angled prism to assist in unobtrusive visualisation.

In a still further embodiment, the invention relates to a scope for examining or surgically treating a part of the body (for example a difficult to access part of the body such as the ear, nose or throat) comprising:
a probe;
at least one visualiser on the probe,
an optical configuration, and
a light source

wherein the optical configuration includes lens elements which are adjustable to focus images. Suitably, the optical configuration comprises combinations of an adjustable lens and sensor element, a fixed front element, and adjustable sensor, a rod lens, an adjustable front lens and an angled front lens.

In another embodiment, the invention relates to a scope for examining or surgically treating a part of the body (for example a difficult to access part of the body such as the ear, nose or throat) comprising:
a probe;
at least one visualiser on the probe,
an optical configuration, and
a light source

wherein the optical configuration includes mechanically coupled lens elements.

The invention also relates to a scope for examining or surgically treating a part of the body (for example a difficult to access part of the body such as the ear, nose or throat) comprising:
a probe;
at least one visualiser on the probe,
an optical configuration, and
a light source

wherein the optical configuration is a staged focus optical configuration. Preferably, the staged focus optical configuration comprises a solenoid and spring-operated staged focus optical configuration. Alternatively, the staged focus optical configuration comprises a ratchet mechanism or a friction controlled mechanism.

The articulatable visualiser and in particular the rotatable visualiser is particularly beneficial in minimal invasive surgery as the eyeball camera can point in different directions including straight ahead (0°) and rotated to different angles. In ear surgery, this allows a surgeon to see around the corner of the ear canal or up into the epitympanum/attic in the middle ear.

The self-cleaning module of the scope of the invention removes blood and other debris that can adhere to the front lens, so that the scope or probe do not need to be removed during surgery for cleaning thus avoiding disruption or delay during surgery.

The optical configuration of the scope of the invention is configured to allow for variable focusing and refocusing as required e.g. when the front lens is positioned above a surgical site to observe operation of tools or, at other times, when the front lens is pushed up close to observe tissue structure.

The scope of the invention, and in particular, the probe and articulatable visualiser in the form of the eyeball camera can be easily disassembled for cleaning and re-use.

The skilled person will appreciate that except where mutually exclusive, a feature described in relation to any one of the above aspects may be applied to any other aspect.

### BRIEF DESCRIPTION OF THE DRAWINGS

The invention will now be described, by way of example only, with reference to the accompanying drawings in which:

FIG. 1(a) is a perspective view from below and one side of a first embodiment of a scope of the invention made up of a probe and a uniform articulatable visualiser on the probe in the form of a rotatable camera eyeball operable by a visualiser orienting mechanism;

FIG. 1(b) is an enlarged perspective view from below and one side of the eyeball of FIG. 1(a);

FIG. 2 is an exploded perspective view from below of the scope of FIGS. 1(a) and 1(b);

FIG. 3 is a side elevation of the scope provided with a belt-driven visualiser orienting mechanism with the direction of rotation of the belt and eyeball indicated by arrows;

FIG. 4 is a side elevation of the scope provided with a hydraulic visualiser orienting mechanism with the direction of rotation of the eyeball and hydraulic fluid indicated by arrows;

FIG. 5 is a side elevation of the scope provided with a centrifugal or pelton wheel liquid visualiser orienting mechanism with the direction of rotation of the eyeball and liquid indicated by arrows;

FIG. 6 is an enlarged side elevation of the visualiser self-cleaning module of the eyeball of FIG. 1 with the direction of rotation of the eyeball indicated by an arrow;

FIG. 7(a) is a perspective view from above and one side of the scope having three internal channels in the probe made up of front and rear eyeball rotation channels and a data/cleaning channel and two external power channels for transmitting power to the eyeball;

FIG. 7(b) is a side elevation of the scope of FIG. 7(a);

FIG. 8 is a side elevation of the scope of FIGS. 1 to 7(b) in which the probe is provided with a hinge to further assist with visualising the ear;

FIG. 9a is an exploded view of the eyeball;

FIG. 9b shows schematic views of communication modules provided in the camera and probe;

FIG. 10 is a side view of another embodiment of the scope of the invention in which the image sensor is relocated from the eyeball into the probe and the eyeball is rotated to a 0° position;

FIG. 11 is a side view of the scope of FIG. 11 in which the image sensor is rotated to a 45° position;

FIG. 12 is a side view of the scope of FIG. 10 in which the image sensor is rotated to a 120° position;

FIG. 13 is a side view of another embodiment of the invention similar to the embodiment of FIGS. 10 to 12 but with an alternative lens configuration rotated to a 0° position;

FIG. 14 is a side view of the scope of FIG. 13 in which the image sensor is rotated to a 45° position;

FIG. 15a is a schematic representation of a further embodiment of the invention similar to the embodiments of FIGS. 10 to 14 in which the image sensors are stacked to save space and reduce the profile of the probe;

FIG. 15b is a schematic representation of a focusing mechanism provided in the camera;

FIG. 15c is another schematic representation of the focusing mechanism in which vents are provided;

FIG. 15d is a schematic representation of another embodiment of the vents of FIG. 15c;

FIG. 15e is a schematic representation of an embodiment of the scope having movable cleaning members provided on the camera;

FIG. 15f is a schematic representation of an embodiment of the scope having movable cleaning members on the probe;

FIG. 15g is a cross sectional view through the eyeball camera of an embodiment;

FIG. 15h is another cross sectional view through the eyeball camera shown in FIG. 15g;

FIG. 15i is a cut away version of the view shown in FIG. 15h;

FIG. 15j is a side view of the distal end of the scope showing the eyeball camera;

FIG. 15k shows schematic representations of various embodiments of the orienting mechanism provided to rotate the eyeball camera;

FIG. 15l shows a schematic representation of an alternative embodiment of the self-cleaning module;

FIG. 15m is a schematic representation of an embodiment of the scope having a wireless power transmission system;

FIG. 16(a) is a side elevation of a further embodiment of the scope in which the articulatable visualiser is a tiltable camera controllable with a directional lever and the scope is provided with a stabilizer in the form of a speculum;

FIG. 16(b) is a side elevation of the endoscope of FIG. 16(a) in which the directional lever is replaced by a directional twist knob;

FIG. 17(a) is a side elevation of a further embodiment of the scope similar to the scope of FIGS. 16(a) and (b) but in which the probe is curved to conform with the speculum and provide free access space for a surgeon and the orientation of the camera is controllable via a directional wheel and the probe is slidable to adjust the depth of the probe;

FIG. 17(b) is a side elevation of the endoscope of FIG. 17(a) in which the orientation of the camera is adjustable via a slider;

FIGS. 18(a) to 18(b) are side elevations of a further embodiment similar to that of FIGS. 16 to 17 but in which the probe is also slidable with respect to the speculum;

FIG. 19 is an enlarged cross-sectional view of a further embodiment of the invention in which the visualiser orienting mechanism is an external spring-operated living hinge to facilitate orientation of the camera;

FIG. 20 is an enlarged cross-sectional view of an internal spring-operated living hinge of the probe orienting mechanism to further facilitate orientation of the camera;

FIG. 21 is an enlarged cross-sectional view of the probe orienting mechanism in which a spring-operated double living hinge of the probe facilitates orientation of the camera;

FIG. 22 is a side elevation of a further embodiment of the scope but in which the endoscope is further provided with an optical configuration that includes an angled prism adjacent the camera to assist in unobtrusive visualisation;

FIG. 23 is an enlarged cross-sectional view through optical configurations of the probe showing the lens elements of the optical configuration which are movable to focus images;

FIG. 24 is an enlarged cross-sectional view of an alternative optical configuration in which the optical configuration includes mechanically coupled elements;

FIG. 25 is an enlarged cross-sectional view of yet an alternative optical configuration in which the optical configuration includes a solenoid and spring-operated staged focus arrangement;

FIG. 26 is an enlarged perspective view from above and one side of the camera on the tip of a probe in which the scope is provided with four light emitters surrounding the camera;

FIG. 27 is a schematic representation of various light emitter arrangements at the camera of the scope, and

FIG. 28 is a schematic representation of light being transmitted through the speculum to the probe camera.

FIG. 29 is a side elevation of the scope provided with a double roller-driven visualiser orienting mechanism; and

FIGS. 30a and 30b are side elevational views of a visualiser self-cleaning module comprises an elastomeric sheath.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human.

### EXEMPLIFICATION

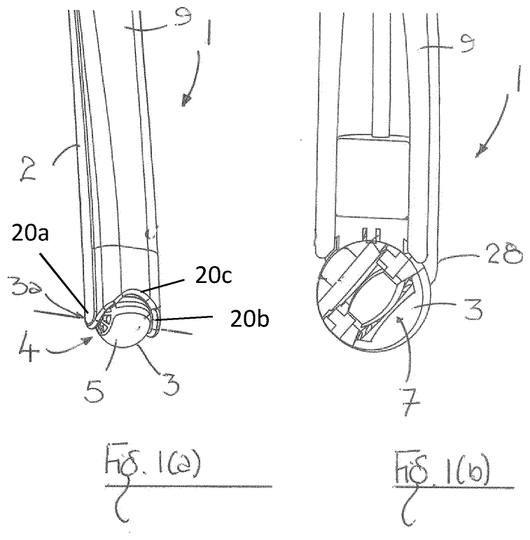

As shown in FIGS. 1 to 9 of the accompanying drawings, a scope of the invention is generally indicated by the reference numeral 1 and is made up of a probe 2 and an articulatable visualiser 3 which can be oriented/tilted and/or rotated about a visualiser axis 3a via an orienting mechanism 4 for optimal viewing of the ear. In the present embodiment, the articulatable visualiser 3 is a substantially spherical rotatable eyeball-like image sensor or camera 5. However, as will be appreciated by those skilled in the art, in other embodiments, the articulatable visualiser 3 can have other non-spherical eyeball shapes provided the articulatable visualiser has a uniform profile about its articulation or rotational axis 3a. The scope 1 is also provided with a light source 42 which can be incorporated into the camera 5 as in the present embodiment or separate from/adjacent to the camera 5 as shown for example in FIGS. 26 to 28. The scope can be an otoscope for examining ears or a surgical endoscope which allows for bi-manual diagnosis and surgical techniques whilst benefiting from the advantages associated with endoscopic visualisation of the ear.

The scope 1 is further provided with a self-cleaning module 6 for cleaning detritus from the eyeball camera while in use without requiring removal of the scope from the ear. More than one self-cleaning module may be provided, which may be the same or different. This shall be explained more fully below. The eyeball camera 5 is also fitted with an optical configuration 7 to optimise images from the eyeball camera 5 made up of lens elements 8 and a focusing mechanism 41 for controlling the lens elements.

The probe 2 is made up of an elongate tubular body 9 defining an open mouth 10 at its distal end for holding the eyeball camera 5. Externally, the elongate tubular body 9 is provided with first and second oppositely disposed side channels 11, 12 respectively for receiving respective detachable first and second side panels 13, 14. The first and/or second side channels 11, 12 serve to house power lines 15 for powering the eyeball camera 5 and terminate at power pins 16 insertable in the eyeball camera 5.

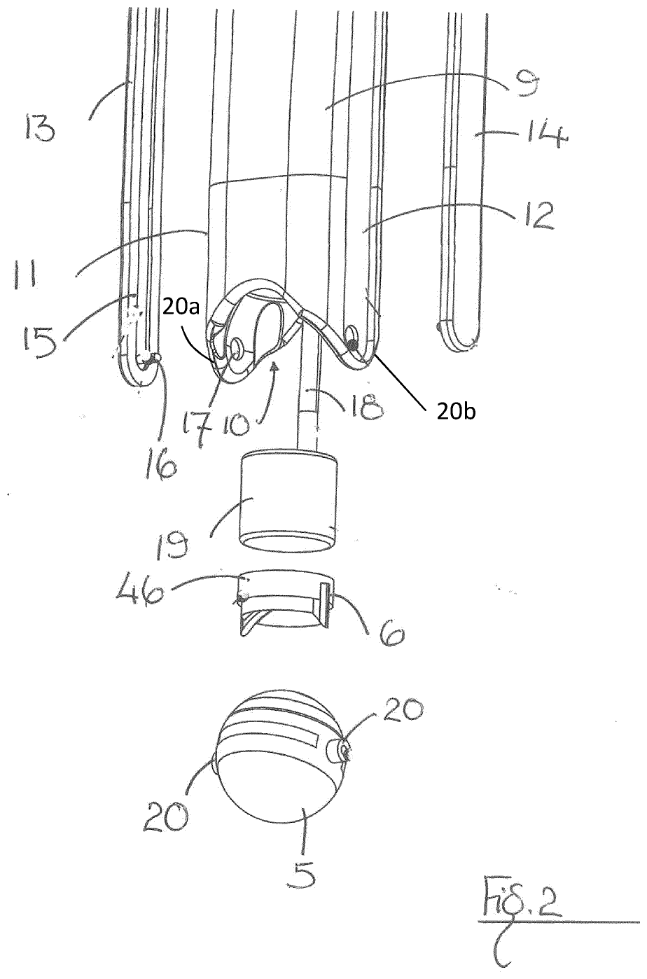

At the mouth 10, the probe tubular body 9 is shaped to define two oppositely disposed axis holes 17 (e.g. receptacles) for supporting the eyeball camera 5 in the mouth 10 at two oppositely disposed axis pins 20, which define the visualiser axis 3a and extend laterally outwards from the eyeball camera 5. The probe body 9 comprises a pair of tabs 20a, 20b that support the camera 5. The tabs 20a, 20b form a structure on which the axis holes 17 are located. The tabs each extend in a distal direction from the distal end of the probe body 9 as can be seen in FIG. 2. The tabs 20a, 20b are opposed to each other about a central longitudinal axis of the probe body 9 such that the visualiser axis 3a extends between the tabs. The probe body further comprises distally opening notches (one or which can be seen in FIG. 1 labelled 20c) that are defined between the pair of tabs 20a, 20b. The notches are defined on opposing sides of the probe body between the tabs 20a, 20b such that the notches are mutually aligned on an axis that is substantially perpendicular to the visualiser axis 3a. The notches form respective cut-out portions of the distal end of the probe body relative to the tabs. This allows the range of view of the camera to be increased by removing material from the probe body that would otherwise block the field of view, yet still allowing the camera to be supported to rotate around the rotational axis. In other embodiments, the axis holes and pins may be reversed so that the holes are provided on the camera and the axis pins on the tabs. In yet other embodiment, only one tab may be provided to support the camera in a cantilever arrangement.

A data cable 18 extends through the tubular body 9 from the eyeball camera 5 via a wireless communicator module 19.

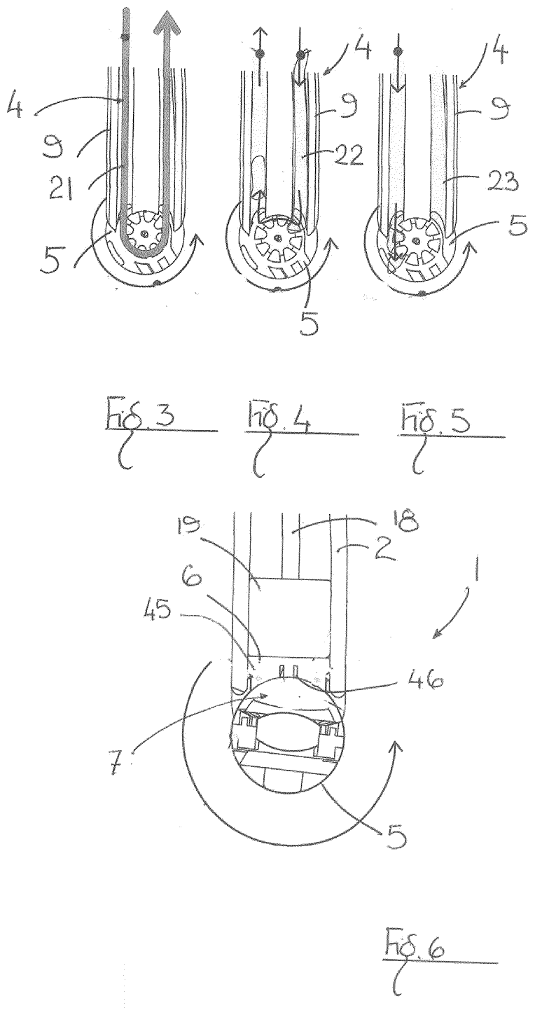

The orienting mechanism 4 for rotating the eyeball camera 5 in the mouth 10 of the probe 2 can function in a number of ways as desired. For example, as shown in FIG. 3, the orienting mechanism 4 can be a belt- or band-driven orienting mechanism 21 while, as shown in FIG. 4, the orienting mechanism 4 can be a hydraulic orienting mechanism 22 operating similar to a vane or gear pump. Alternatively, as shown in FIG. 5, the orienting mechanism 4 can be a air orienting mechanism 23 operating in a manner similar to a centrifugal pump or pelton wheel.

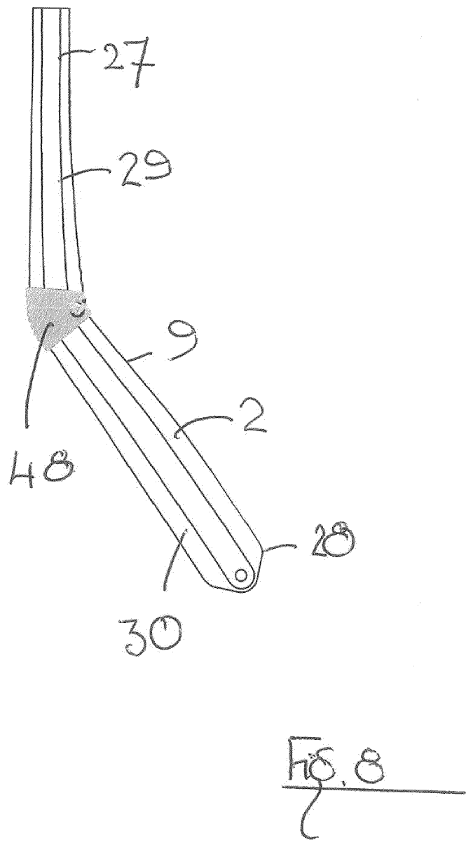

As shown particularly in FIG. 7(a), the orienting mechanisms 4 of FIGS. 3 to 5 can be accommodated in first and second internal channels 24, 25 defined in the probe elongate body 9. In addition, the probe elongate body 9 can be provided with an internal data and/or cleaning channel 26 for receiving the data cable 18 and/or carrying fluid and detritus to and/or from the self-cleaning module 6. The data channel 26 is large at the distal end 28 where it holds the wireless communication module 19 and cleaning module 6 while the first and second internal channels 24, 25 are stacked at the distal end 28. Proximally from the distal end 28 the first and second internal channels 24, 25 reorientate and resize as shown in 7(a) to allow for a more narrow profile probe towards the proximal end 27 as indicated by the reference numeral 29 than towards the distal end 28 as indicated by the reference numeral 30, thus making it easier to manoeuvre tools past the probe as shown in FIG. 7(b). Moreover, the resultant narrow profile 29 allows for flexible bending of the probe 2 at a hinge 48.

In other embodiments, additional internal channels can be provided to supply liquid air and vacuum to integrate with the cleaning module 6 and for use in surgery. Alternatively or in addition, a working channel can be provided for tools such as a needle for injection.

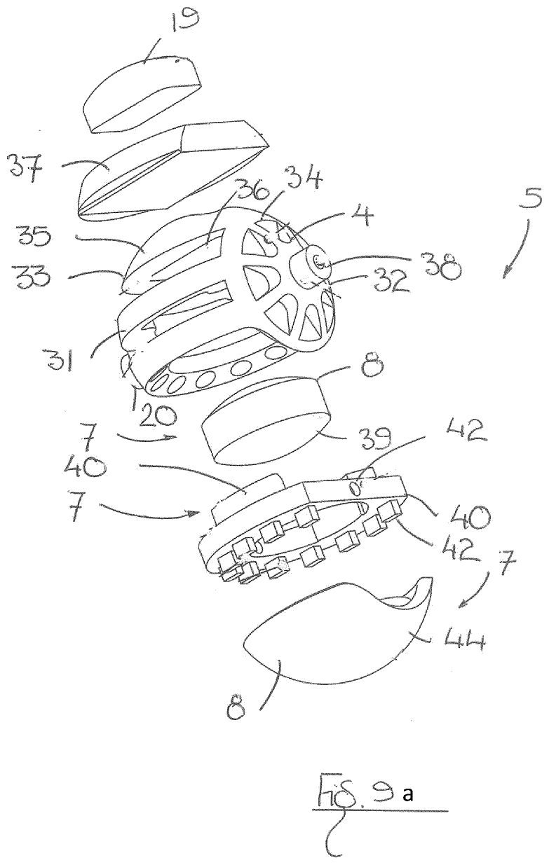

As shown particularly in FIG. 9a, the eyeball camera 5 of the present embodiment is made up of a rotatable cage-like housing 31 having a first side 32 and an opposite second side 33. The eyeball camera 5 is received within the open mouth 10 at the distal end of the probe as shown in FIG. 1. The housing 31 is provided with an element of the orienting mechanism 4 in the form of a wheel 34 formed at the second side 33 of the housing 31. The first side 32 is provided with a first axis pin 20 as previously described while the wheel is provided with a second axis pin 20 defining the rotational axis 3a. The wheel 34 is operable by the orienting mechanism 4 to rotate the housing 31 on the axis pins 20 and hence the eyeball camera 5. The axis pins 20 each have power sockets 38 for receiving power from the power pins 16. The cage-like housing 31 is further provided with ribs 35 extending between the first and second sides 32, 33 which define cage board slots 36 therebetween for receiving and holding functional boards of the eyeball camera 5 such as an image sensor board 37.

As can be seen in the Figures, the eyeball camera 5 is mounted such that part of the housing (the housing including the outer lens 44) is disposed distally beyond the distal end of the probe body. More specifically, a portion of the housing of the camera extends distally beyond a distal end of the tabs. An outer surface of the camera 5 thus forms an outer surface of the scope and is cleaned by the self-cleaning module.

The image sensor board 37 is arranged to generate image data that is transmitted from a wireless communication module located within the housing of the camera 5 to wireless communication module 19 provided in the probe. The communication module is configured to transmit and/or receive image data at a frequency equal to or in excess of 2.4 GHz. This allows a smaller antenna to be used. The skilled person will understand other frequencies can be used. The communication module provided in the probe may be less than 30 mm from the camera.

Embodiments of the wireless communication modules provided in the camera 5 and the probe 2 are shown in FIG. 9b parts (1) to (4). In each of these embodiments, the camera 5 comprises an image sensor 37 in communication with an electronics unit 200. The electronics unit may be a single unit as shown in the figures, or distributed components (it may correspond to the mainboard 40). The electronics unit comprises a main control unit (MCU) 201a. The MCU 201a may be provided on or correspond to the main board 40 described elsewhere herein. The camera 5 comprises a wireless communication module in communication with the MCU 201a. The wireless communication module comprises an RF 201b unit provided in the electronics unit 200 and a camera antenna system 202, 204. The MCU 201a is configured to control the image sensor 37 and the antenna system 202, 204. For example, the MCU 201a may be configured to control the RF transmission rate. It may select the framerate and resolution of the image sensor and bandwidth of the RF transmission. The image sensor is configured to send raw image data serially using MIPI/LVDS protocol or the like to the MCU. The image sensor control signal may come from the MCU in order to control the sensor. The RF unit may be configured to control the transmission signal power and antenna matching circuitry as described later.

The probe 2 comprises a processing unit 206 operatively coupled to a wireless communication module. The processing unit 206 is configured to process images received from the camera. The wireless communication module comprises a second RF unit. The second RF unit comprises a probe antenna system 208, 210 configured for communication with the camera antenna system 202, 204. The wireless communication module provided in the probe 2 is located at the distal end of the probe elongate body 9 close to where the camera 5 is mounted. The wireless communication modules (e.g. their antenna systems) may be less than 25 mm apart, preferably between 5-10 mm apart. The wireless communication module of the probe is connected via a cable 18 to allow data to be transmitted from the scope. The RF unit and antenna system forming the wireless communication module and the processing unit 206 may be provided in a probe electronics unit as shown in the figures. This may be a single unit, or formed by distributed components.

In the embodiment shown in FIG. 9b part (1) the antenna system provided at the camera comprises an omnidirectional antenna 202 in the form of a monopole antenna. The antenna system provided in the probe is a single directional antenna configured to receive an RF signal from the camera antenna 202 in a single direction. The direction of the single directional antenna may be pointing along a longitudinal axis of the probe out of its distal end as shown in the Figures. Other orientations may be used. The monopole antenna caters for the need of the different orientations of the camera relative to the probe while still maintaining suitable signal strength. The use of a single directional antenna at the probe may allow greater signal strength to be received from the camera. In this embodiment, the wireless communication may preferably be used only for transmission from the camera to the probe. In any of the embodiments described herein, the directional antenna may be an embedded antenna or may be a trace provided on a PCB forming part of the antenna electronics. The directional antenna in any embodiment may be a MIFA or IFA type antenna.

In the embodiment shown in FIG. 9b (2) the probe antenna system 210 comprises an omnidirectional antenna (e.g. a single monopole antenna). The camera antenna system 202 comprises a monopole antenna similarly to the embodiment described above. In this embodiment, a one way communication may preferably be provided with the probe antenna system receiving signals from the camera antenna.

In the embodiment shown in FIG. 9b (3) the camera antenna system 204 comprises a two or more directional antenna (three are shown in the figures, however there may be only two, or more than three). The probe antenna system comprises a single directional antenna. In this embodiment, the camera is configured (e.g. the RF module and/or the MCU located in the camera are configured) to select one of the directional antennas in the camera that is closest to the probe antenna according to the relative position of the camera. This can be seen in FIG. 9b (4). This may reduce the amount of radiation being transmitted to the patient by the wireless communication modules. The communication between probe electronics and eyeball camera 5 may be bidirectional (full duplex or half duplex) in this embodiment. The camera may be configured to transmit raw (i.e. unprocessed) image data from camera to the probe via the wireless communication modules. This means that imaging processing circuitry is not required in the camera, allowing it to be made smaller. In other embodiments, images may be processed at a processing module within the camera.

In other embodiments, other means for transmitting image data from the camera 5 may be used. In other embodiments, a data cable may be coupled directly to the housing of the eyeball camera 5 (as will be described later) and extend within the housing to couple to the image sensor.

In other embodiments, image data is transmitted via at least one of the axis pins 20. The image data may be transmitted along with electrical power for the image sensor via one or both of the axis pins. The image data may be transmitted at the same time as the electrical power by modulating the electrical power supply.

The eyeball camera 5 has an internal focusing lens 39 forming part of the lens elements 8 of the optical configuration 7 together with a main board 40 provided with a focus mechanism which also forms part of the optical configuration 7. These components are mounted within the housing as can be seen in FIG. 9a. The eyeball camera 5, and in the present embodiment the main board 40, also has a light source 42 in the form of light emitters 42 on an opposite face thereof for illuminating the ear and oppositely disposed power pin contacts 43 for receiving power at the power sockets 38. The eyeball camera 5 also has an external curved front lens 44 also forming part of the lens elements 8 of the optical configuration 7. The curved front lens may be a window element that has minimal (or no) optical/magnifying power as discussed later such that it acts as a window rather than a lens. The front lens or window forming part of the housing of the camera 5 as can be seen in FIG. 9a. The housing is thus generally spherical as can be seen in the figures, and has a circular profile about its axis of rotation. In other embodiment the housing may have other shapes, and may be cylindrical as described elsewhere herein.

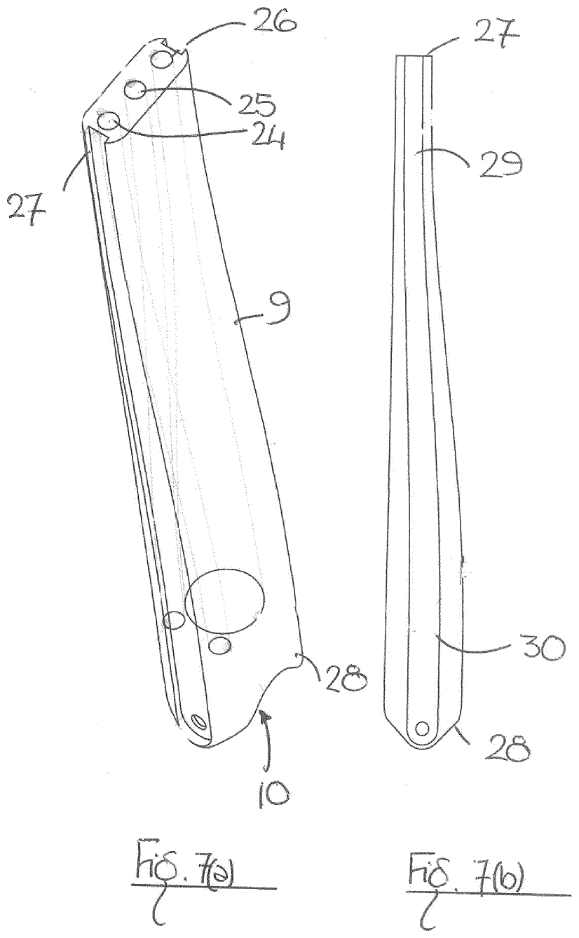

The self-cleaning module 6 (see FIGS. 2 and 6) is made up of a cleaning element in the form of a ring-like body 45 provided with cleaning blades 46 and a soft cleaning material 47 all shaped and contoured to clean the curved front lens 44 of the eyeball camera 5. The cleaning element is positioned on a proximal side of the housing of the camera 5 as can be seen in FIG. 2. The cleaning element is offset laterally (i.e. offset with respect to the longitudinal axis of the probe) with respect to the housing/camera. The cleaning element is located at the mouth 10 of the probe body such that it is disposed between the tabs 20a, 20b. This allows it to contact the outer surface of the housing for cleaning. In other embodiments, the cleaning module 6 (and its associated cleaning element(s)) may have any other suitable location so that it may provide cleaning of the outer surface of the camera 5. The cleaning element may, for example, contact any point on the circumference of the camera about its rotational axis. It may in some embodiments be located distally relative to the camera.

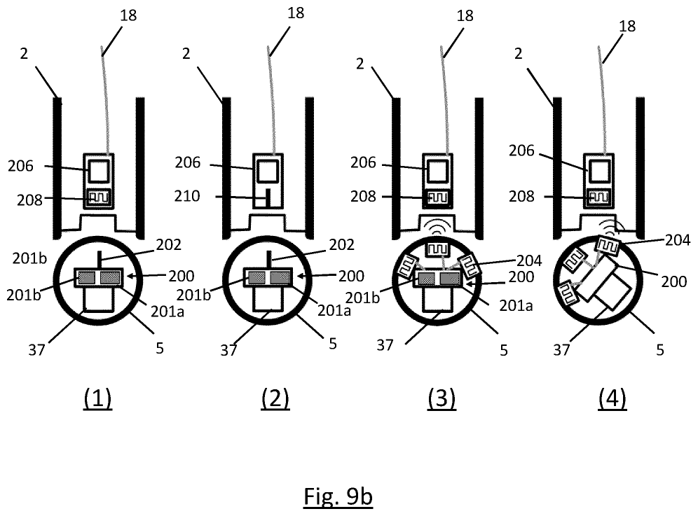

FIGS. 10 to 14 show another embodiment of the scope 1 of the invention in which the image sensor board 37 is relocated from the eyeball camera 5 as shown in FIGS. 1 to 9a into the probe body 10. The eyeball camera 5 can be rotated as shown in the drawings while the self-cleaning module 6 serves to clean the eyeball camera 5 as previously described.

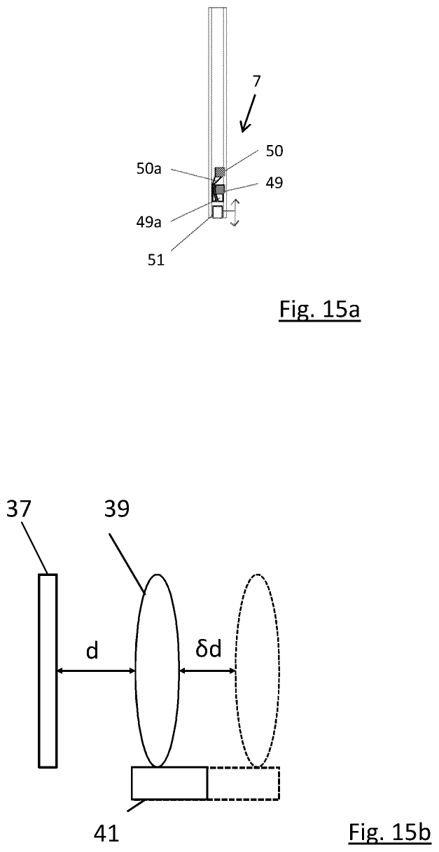

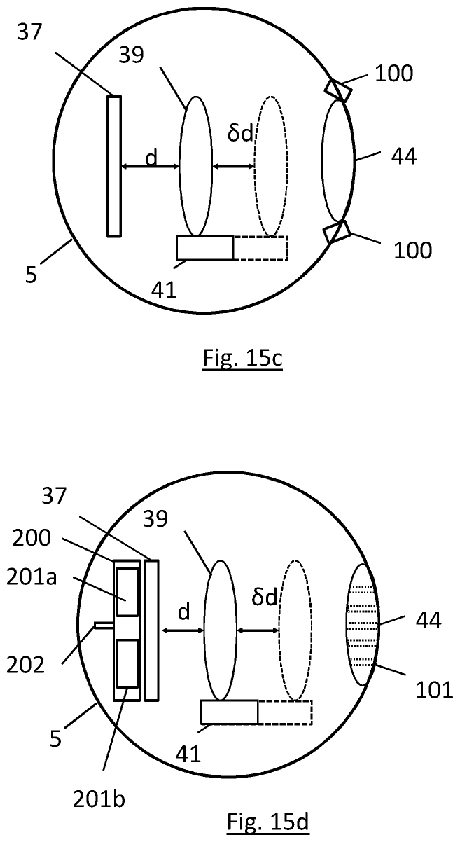

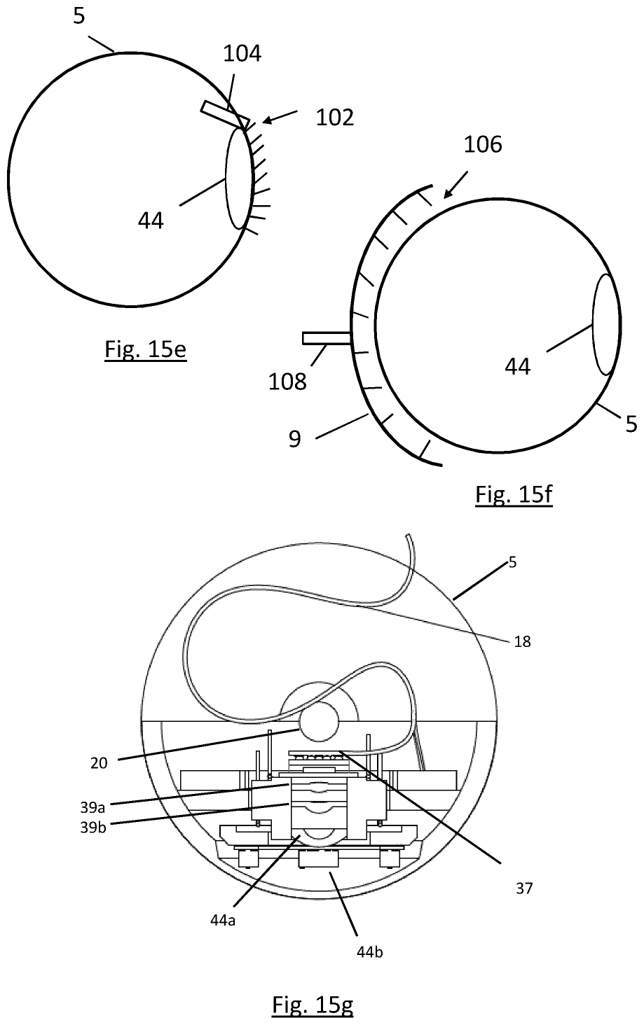

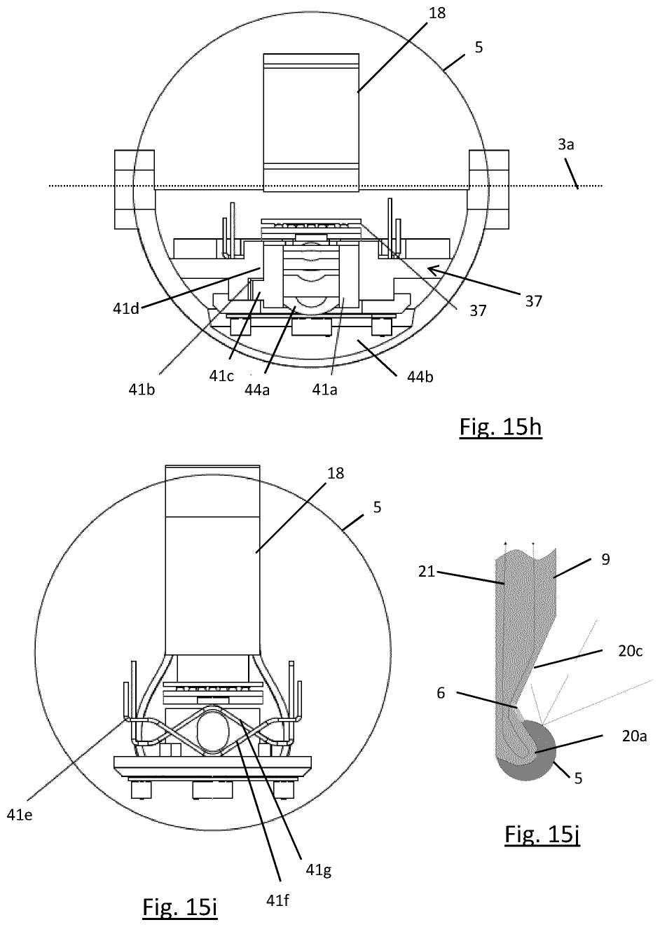

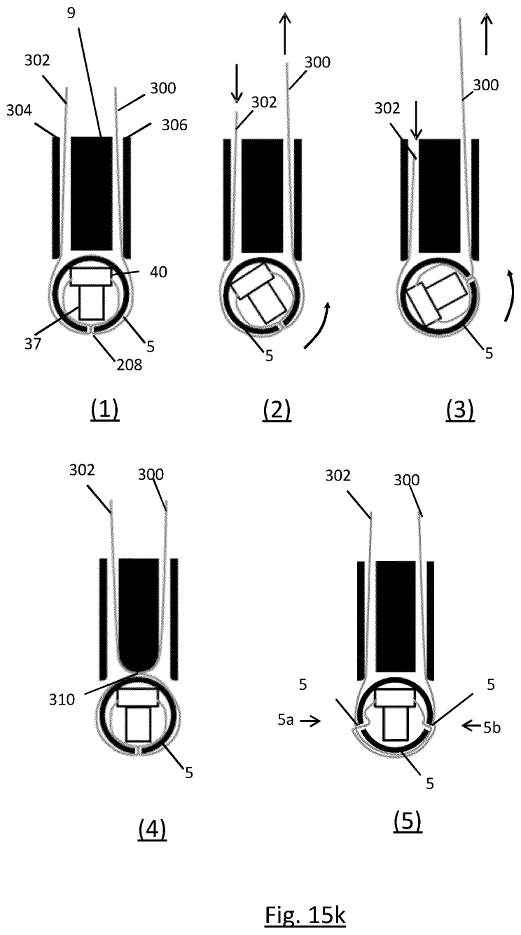

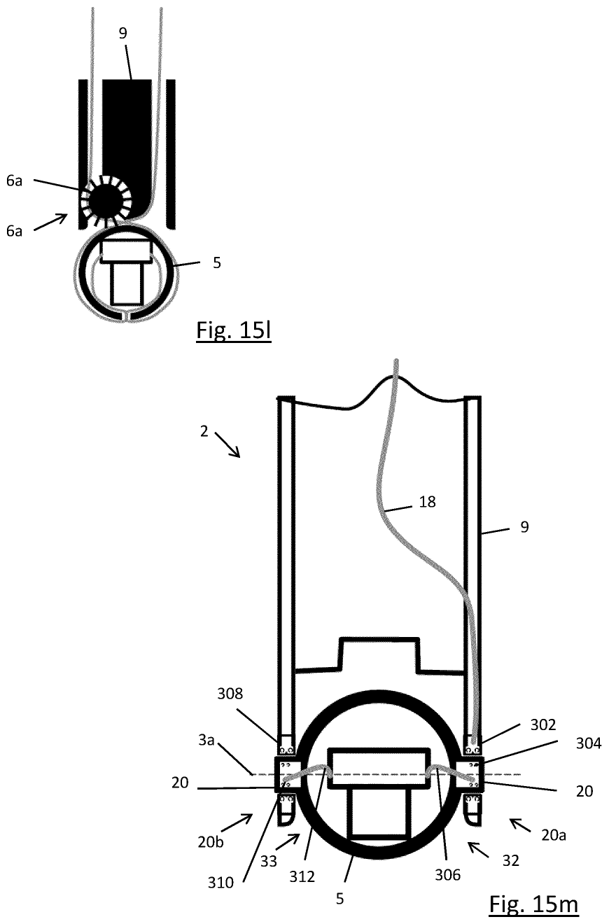

FIG. 15a shows a schematic representation of a further embodiment of the invention in which the image sensor boards 37 are stacked to save space and reduce the profile of the probe 1 so that the scope 1 can fit into different lumens in the body. As the image sensors are stacked to save space and the lenses deliver a spatially separated image to each sensor, image processing software can deliver this image in a binocular view to give depth perception with a low-profile probe. More particularly, as shown in the drawing, the stacked optical configuration 7 is made up of first and second stacked fixed image sensors 49, 50 and corresponding first and second lens elements 8 directing light to the image sensors 49, 50 together with a slidably adjustable front lens 51.

In one embodiment aspherical lenses can be used to bypass the first fixed sensor 49 in an efficient manner e.g. crescent profile lenses (in plan view).

In use, the eyeball camera 5 of FIGS. 1 to 9a is held in the probe 2 by the axis pins 20 which are held in the axis holes 17. This allows the eyeball camera 5 to rotate fully 360° on the axis 3a—the rotation has the dual purpose of reorienting the camera direction and cleaning the front lens 44. In some embodiments an unrestricted 360° may not be provided, for example, if a cable is attached to the camera for power and or data transmission.

In use, the probe 2 is inserted into the internal ear structure of a patient. The housing is then rotated relative to the probe body 9 to cause rotational movement of the image sensor 37 relative to the internal structure of the ear. As shown in FIGS. 10 to 12 discussed later, the housing (and therefore the camera 5) is rotated in use through at least 90° relative to the probe body 9 while viewing the internal ear structure throughout the rotational movement.

The housing (and therefore the camera 5) may be rotated from a first position in which the image sensor 37 faces a distal direction (e.g. FIG. 10) to a second position in which the image sensor 37 faces a proximal direction (e.g. FIG. 12). Once moved from the first to the second position the housing may be rotated back again. Rotating the housing from the first position, to the second position and back to the first position may comprise rotating the housing 360° about its rotational axis.

Rotating the housing may alternatively comprise rotating the housing through at least 120° relative to the probe body during use. During use the front lens 44 or internal lens may be focused by the focusing mechanism.

In use, the outer surface of the housing of the camera 5 (e.g. the outer lens 44 or window) may be cleaned by rotation relative to the self-cleaning module (e.g. relative to the cleaning element of the self-cleaning module). Cleaning the outer surface of the camera 5 comprises wiping the outer surface of the housing with the cleaning element of the self-cleaning module 6. This may be done by rotating the housing from the first position to the second position defined above, and returning to the first position. The second position may be a position in which the image sensor 37 faces generally perpendicularly relative to the central longitudinal axis of the probe body.

Accordingly, the front lens 44 is cleaned by rotating 360°, as shown in FIG. 6, thus wiping the front lens 44 with the self-cleaning module 6 which is held in the probe 2. The self-cleaning module 6 consists of materials to clean the lens such as the flexible blade 46 and soft material 47. In another variant of the invention, the cleaning module 6 can be integrated with internal vacuum and liquid channels in the probe 2.

Because the eyeball camera 5 is only connected to the probe at the axis pins 20, there is limited space for power and data pins/contacts or cables especially in smaller size implementations. Accordingly, only one power pin contact 43 is delivered through each axis pin 20 so that power is delivered through these pin contacts 43 and data from the image sensor can be delivered using a wireless technology between the wireless communicator 19 from which the data cable 18 delivers data to an image processor outside of the scope 1.

The wireless communicator 19 of the eyeball camera 5 can also measure the angle of rotation which can be displayed to the user. In another embodiment, data can also be delivered by signal modulation in the two power pins 16. In this case, an image processor must separate the data signal from the power to display an image.

As shown in FIGS. 3 to 5, the eyeball camera 5 can be controlled using a belt or band orienting mechanism 21, a hydraulic orienting mechanism 22 or a liquid orienting mechanism 23 in a fashion similar to a vane or gear pump. Fine controls for systems using hydraulic actuation can be provided through manual push buttons on the scope 1. In other embodiments, the orienting mechanism can be a motor built into the probe 2 or the eyeball camera 5. Alternatively, a wheel or gear in the probe 2 actuates the eyeball camera 5 or a wire can be used in a crank fashion to rotate the eyeball camera 5.

In the embodiment shown in FIG. 3, the belt orienting mechanism comprises a belt coupled to a drive shaft positioned at a proximal end of the probe and a driven shaft acting on the housing of the camera. The belt may be wrapped around the drive shaft and/or the driven shaft at least twice. This may provide improved engagement between the drive shaft(s) and belt so that slippage does not occur when the scope is used in water. In other embodiments, other arrangements of belt may be provided. The drive shaft may be operatively coupled to a stepper motor (not shown in the figures) for rotating the drive shaft, and in turn the driven shaft and the camera housing.

In other embodiments of the invention, there are a different number of electrical boards and the functions are located between them.

The internal focus lens 39 is held in position by the focusing mechanism 41 of the optical configuration 7. The focusing mechanism is mounted within the housing of the camera 5. The focusing mechanism 41 positions the lens between the front lens 44 and the image sensor board 37 and repositions the focus lens 39 to change focus of the eyeball camera 5. The focusing mechanism 41 is electromechanically operated. It may, for example, comprise a microelectromechanical system.

In one variant, the focusing mechanism 41 can employ electromagnets and in other embodiments, the focusing mechanism 41 can employ bimetal linkages. Alternatively, harmonics can be used to control linkages which move the focusing lens 39. In yet other embodiments, the focusing mechanism comprises a shape memory metal component configured to focus the lens as described in more detail elsewhere herein.

FIG. 15b illustrates another embodiment of the focusing mechanism, which may be used in combination with any other embodiment described herein. In this embodiment, the focusing mechanism is arranged to vibrate the focusing lens 39 (or at least one or all of them if more than one is provided) so that the focusing distance of the focusing lens(es) relative to the image sensor 37 (labelled ‘d’) varies over a range of motion (labelled ‘δd’). The focusing lens 39 is shown in FIG. 15b in solid lines at the extent of its motion closest to the image sensor 37, and in broken lines at the other extent of its motion furthest from the image sensor 37. The focusing lens 39 may be mounted to a MEMs or piezo electric device forming part of the focusing mechanism 41 or the like to provide the desired range of motion.

The frequency and amplitude of the vibration of the focusing lens may be controlled at a predefined amount. This may be a constant frequency and amplitude to provide a constant range of motion of the focusing lens. The image sensor is arranged to sample the image data generated in order to generate focused images by selecting frames corresponding to the desired focal point, and use them to form a video image. The other non-focused frames may be discarded. This allows a varying focus to be implemented without relying on positional accuracy of the focusing mechanism controlling the distance between the focusing lens and image sensor. In some embodiments, the image processing may be performed at the image sensor (e.g. by a processor within the camera 5). This may reduce the amount of data transmitted from the camera 5 (by the wireless communication module or cable). In other embodiments, the image processing may be performed outside of the scope using the complete set of image data generated by the image sensor.

In other embodiments, the frequency (X Hz) and amplitude (δd) at which the lens is vibrated and the image sampling rate (Y Hz) may be varied. By varying the ratio Y/X the number of images N per transit of the lens can be varied. For each transit, images corresponding to focal points N can be selected, with the others discarded. By optimising δd, X, Y and N a focused video stream can be generated (e.g. using a suitable deconvolution algorithm).

The vibration of the focusing lens may also be used to aid cleaning of the outer surface of the camera (e.g. the lens 44 or window of the camera housing). In one embodiment, the focusing mechanism is selectively mechanically coupled to the outer lens 44 or window by a switchable dampener. The damper is arranged to switch between a damping condition in which vibration of the outer lens 44 is damped (e.g. during normal operation) and a locked condition in which vibration is not damped and the vibration of the focusing mechanism is transmitted to the outer lens 44 or window. This vibration of the outer lens 44 or window aids cleaning by causing accumulated dirt/debris to be removed.

In some embodiments, the image sensor may be movable relative to the focusing lenses, which may be fixed relative to the camera. In this embodiment, the focusing mechanism is arranged to vibrate the image sensor to achieve the same focusing described above. The skilled person will understand that anything described herein in relation to moving/vibrating focusing lens(es) can also apply to the image sensor (or both).

In other embodiments, the transmission of the vibration of the focusing lens 39 to the outer lens 44 or window is controlled by tuning the vibration frequency of the focusing lens to the resonant frequency of the outer lens/window. During normal operation the focusing mechanism may be arranged to vibrate the focusing lens at a first frequency that is different from the resonant frequency of the outer lens 44/window. This allows the focus to be varied as described above, but without the outer lens/window vibrating. The focusing mechanism may be arranged to switch the frequency at which the focusing lens 39 is vibrated to a second frequency that is the same or about the same as the resonant frequency of the outer lens/window such that it also vibrates. This aids cleaning.

In yet other embodiments, vibration of the focusing lens 39 may induce an air flow over the outer lens/window to provide a cleaning effect. These may be used in combination or separately from the cleaning vibration described above. FIG. 15c illustrates an embodiment in which the eyeball camera 5 comprises vents 100 located adjacent the outer lens 44 (or window). The vents 100 are arranged to provide a stream of air generated by pressure changes within the eyeball camera 5 resulting from the vibration of the focusing lens 39 (e.g. a bellows affect is created by the vibrating lens). The airflow provided by the vents 100 is directed over the outer surface of the outer lens 44 to generate a cleaning effect. Although two vents are shown in the figures other numbers may be provided, such as a single vent or more than two vents.

FIG. 15d illustrates an embodiment similar to that of FIG. 15c. In this embodiment, the outer lens 44 (or window) comprises through holes 101 that act as vents for airflow created by the vibrating focusing lens 39. FIG. 15c also shows schematically the location of the camera antenna system 202 formed from a monopole antenna, the camera MCU 201a and RF module 201b provided in the electronics unit 200. This arrangement may apply to any antenna system and apply to any embodiment of the eyeball camera 5.

FIG. 15e illustrates an embodiment in which the eyeball camera 5 comprises a plurality of movable cleaning members in the form of cilia 102 extending from the outer surface of the lens 44 (or window). The cilia may extend over part or all of the lens/window outer surface. The cilia are arranged to move relative to the outer surface of the lens/window to move debris and so provide a cleaning effect. The movement of the cilia 102 may be powered by the power source provided to the eyeball camera 5. The cilia 102 are arranged to move in a coordinated movement to move material away from the field of view of the lens 44/window. The cilia may be may be made from a transparent material, and/or be suitably thin, to avoid interfering with the field of view. In addition to covering the outer lens 44 the cilia may cover a greater area of the outer surface of the camera 5. In some embodiments, the camera 5 comprises a cleaning fluid supply device 104 which is arranged to supply cleaning fluid to the cilia 102. The cleaning fluid device may comprise a pump arranged to pump cleaning fluid from within the camera 5 to its outer surface at a position adjacent the cilia. Alternatively, the cilia may draw cleaning fluid from the cleaning fluid supply device 104 by capillary action.

FIG. 15f illustrates an embodiment in which a plurality movable cleaning members in the form of cilia 106 are arranged on a surface of the probe body 9 adjacent the eyeball camera 5. The cilia may operate similarly to those described in connection with FIG. 15e. The cilia 106 are arranged to move relative to the probe body (and the eyeball camera). In this embodiment, the cilia 106 are powered by a branch of the power supply to the camera 5 that is located within the probe body 9. The cilia 106 are arranged to move a coordinated movement to move material to a point on the surface of the probe body 9 where they either do not obstruct the view of the camera or can be removed. The probe may further comprise a removal device 108 arranged to remove material collected by the cilia 106. The removal device 108 may be a suction system, and may be located at the centre of the concavity which receives the eyeball camera 5 at the distal end of the probe 2. In other embodiments, the removal device 108 may have any other suitable location. The probe body 9 may further comprise a cleaning fluid device similar to that of FIG. 15e to provide cleaning fluid to the cilia 16 on the probe.

In the embodiment shown in FIGS. 15e and 15f the cilia may be replaced with other types of cleaning member such as rollers that are adapted to move in a coordinated manner to move material and provide cleaning.

The cleaning members and image focusing using the vibrating lens described above, and its associated use for cleaning, can be used in addition to the self-cleaning module described elsewhere herein. They may also be used instead of the self-cleaning module. They may also be used separately from the articulated movement between the eyeball camera 5 and the probe. For example, using a camera that is fixed relative to the probe.

In another embodiment of the invention, the optical configuration can be made up of a variable aperture which can be employed instead of a focusing mechanism 41 deploying lens elements 8 moving relevant to or in conjunction with the image sensor. The aperture may be electromechanical in nature or digital where an opaque filter forms the aperture. Change of the aperture size allows change in the depth of field which can be advantageous because a large depth of field (small aperture) can be used to observe tools from a distance. The depth of field can then be reduced by expanding the aperture, to allow high quality imaging at close range.

The lens elements 8 can be made up of multiple elements to achieve best image quality while, in some embodiments, another variation the outer layer of the front lens 44 is a window to allow in light with minimum distortion.

An example of an embodiment in which the front lens 44 is a window is shown in FIGS. 15g, 15h, 15i. This embodiment is similar to that shown in FIGS. 1 to 9a, with like reference numerals used accordingly. In this embodiment, the camera 5 comprises a window that allows light to pass with negligible (or no) focusing and magnification. The window is aligned with the image sensor mounted within the housing of the camera 5. The front lens 44 is replaced with an internal lens 44a. An optical path is therefore defined from the image sensor 37, through the internal lens 44a and through the window 44b. The optical configuration further comprises focusing lenses 39a, 39b. Although two focusing lenses are provided in this embodiment, there may be only one or greater than two in other embodiments. The focusing lenses 39a, 39b are arranged to focus light from the internal lens 44a onto the image sensor 37. The focusing lenses may be adjusted using the focusing mechanism described anywhere else herein. A fluid filled gap 44b is defined between an inner surface of the window 44ab and an outer surface of the lens 44a. In the present embodiment, the fluid filled gap is an air filled gap. In other embodiments, it may be filled with another suitable light transmitting fluid such as oil. By including the fluid filled gap, a medium of known refractive index is provided at the outer surface of the internal lens 44a. This means that the optical configuration and the focusing of the lens 44a is not changed if the probe is in air or water.

FIG. 15h shows a side view of the embodiment shown in FIG. 15g through a plane that includes the rotational axis 3a. The focusing mechanism 41 comprises a lens stage 41a or train on which the focusing lenses 39a, 39b and the internal lens 44a are mounted. The focusing mechanism 41 is arranged to translate the lens stage 41a relative to the image sensor to focus the image. The lens stage 41a has a range of travel defined by the space 41b between an abutment part 41c of the lens stage and a corresponding fixed abutment part 41d of the focusing mechanism. In the presently described embodiment, the range of travel of the lens stage is 40 microns. This may be adjusted as appropriate for the lenses being used.

In this embodiment, all of the focusing lenses 39a, 39b and the internal lens 44a are mounted to the lens stage 41a and are movable relative to the image sensor 37. In other embodiments, one or more of these lenses may be fixed relative to the lens stage, so that at least one movable lens is provided on the lens stage in order to provide image focusing.

FIG. 15i shows a cut away illustration of the embodiment shown in FIG. 15h. The focusing mechanism comprises a metal memory component as mentioned above arranged to provide movement of the focusing lens/and or internal lens relative to the image sensor. The metal memory component is formed from a shape memory alloy that contracts when a voltage is applied to it. The metal memory component is coupled to the lens stage 41a and a relative fixed part of the focusing mechanism to provide relative motion therebetween upon application of a suitable voltage. In the present embodiment, the memory metal component comprises a first section arranged to cause motion of the lens stage towards the image sensor and a second section arranged to cause motion of the lens stage away from the image sensor. Other arrangements of memory metal component may be provided.

In the embodiment shown in FIGS. 15g, 15h, 15i the cable 18 is connected to the image sensor 37 and extends out of the camera 5 where in extends along the probe body 9. The cable 18 is flexible and has a length suitable to allow rotation of the camera housing relative to the probe body 9.

In some embodiments, flexible optics are used to change the surface profile of the lens elements 7 to change the focus.

If desired, additive manufacturing can be used to 3d print the lenses.

As indicated above, in some embodiments, the articulatable visualiser is not spherical but can be configured differently e.g. a cylindrical or other uniform profiles around its axis 3a. Uniform profiles such as a cylinder or sphere are advantageous in use because the uniform profile doesn't change shape when rotated against tissue and is less likely to cause trauma to tissue. In another embodiment, a window also surrounds the eyeball camera 5 with a profile similar to a U to ensure that the eyeball camera 5 does not cause trauma to tissue.

The camera may have a circumference that is less than 6.5 mm measured through the rotational axis. The camera may have a circumference that is less than 3.5 mm measured through the rotational axis. Other sizes may be possible, and the application is not limited to these examples.

However, in other embodiments, the profile of the articulatable visualiser 3 need not be uniform around its axis 3a.

In another embodiment, the front lens 44 protrudes further than the rest of the eyeball camera 5, to contact with the self-cleaning module 6 when rotated, while the rest of the eyeball camera 5 does not contact the self-cleaning module.

FIG. 15j illustrates an embodiment of the distal end of the probe body 9 showing the mounting of the eyeball camera 5. The eyeball camera 5 is mounted on tabs (one of which is visible in FIG. 15j labelled 20a) as already described with notches between them. In this embodiment, one of the notches 20c forms a larger cut away region. In the cut away region the cross sectional size of the probe body 9 is reduced to form a tapered portion that increases the field of view of the camera 5 when rotated to point the image sensor 37 in a proximal direction. This provides minimal occlusion of the field of view of the camera 5. FIG. 15j also illustrates the belt driven orienting mechanism 21 having a drive band/belt extending along the length of the probe body 9 to drive rotation of the camera 5.

FIG. 15k illustrates another embodiment of the orienting mechanism. This may be used in combination with any other embodiment herein. In this embodiment the orienting mechanism comprises a first cable 300 and a second cable 302, each connected to the camera 5. The first cable 300 is arranged to extend from its point of connection with the camera around the camera and to the probe in a first direction about the rotational axis of the camera. The second cable is arranged to extend around the camera and to the probe about the axis of rotation of the camera in a second direction (opposite the first). The first cable is arranged to rotate the camera in the first direction and the second cable is arranged to rotate the camera in the second direction. As can be seen in the figures, tension applied to the first cable causes rotation of the camera about its rotational axis in the first direction (anticlockwise FIG. 15k). Tension in the second cable causes rotation of the camera in the second direction around the rotational axis (clockwise FIG. 15k). As can be seen in parts (2) and (3) of FIG. 14b pulling the first cable in a proximal direction along the length of the probe body causes anti-clockwise rotation of the camera around the rotational axis. Although not shown in the figures, the cables may each extend to a proximal end of the scope where there may be coupled to a suitable actuation device.

The cables may extend along channels 304, 306 provided in the probe body 9 that are described elsewhere herein, e.g. similarly to the drive belt. The cables both extend through an aperture 208 in the outer housing of the camera 5 and within the housing so that they are coupled to the camera electronics 200 e.g. the main board 40 or other suitable point (e.g. to the MCU).

In the presently described embodiment, the cables 300, 302 extend from the camera 5 at a position at the front of the lens. Specifically, they both extend from a zero degree position in front of the lens that corresponds to the most distal point or the camera when it is in an unrotated position (e.g. with the image sensor pointing along the longitudinal axis of the probe body). This provides a greater range of rotation. The cables 300, 302 may however extend through or be coupled to the camera at any other suitable position around the axis of rotation of the camera. They may in other embodiments be connected to or extend through the outer wall of the camera at different points, e.g. extend through separate apertures. By “extend from” we mean a point at which the cables extend through an aperture in the camera or a point to which they are fixedly connected.

In the embodiment shown in part (3) the first and second cables 300, 302 may cross over each other so that each of the cables extend around opposite sides of the camera 5 compared to the side of the probe body along which they extend. The cables cross at a crossing point 310 aligned with the axis of rotation of the camera. This may allow full 360 degree rotation of the eyeball camera 5.

In other another embodiments the cables may enter the eyeball at two different positions on the circumference of eyeball camera 5, rather than one entry or connection point 208, if the cables travel the long way around the circumference to these entry points, then rotation greater to 360 degrees may be achieved. This is illustrated in FIG. 15k (5).

In the embodiment shown in FIG. 15k (5), the orienting mechanism comprises first and second cables 300, 302 extending from different points around the circumference of the camera about the rotational axis. The points from which the cables extend may be mutually opposed about the visualizer axis 3a of rotation of the camera (e.g. the axis of rotation of the housing). The first cable 300 may extend from a point on the camera on a first side 5a relative to the rotational axis and extend around the camera around an opposing second side 5b of the camera. The second cable 302 may extend from a point on the second side 5b and extend around the first side 5a in the opposite direction. The first and second cables each therefore extend around the distal zero degree position of the camera and overlap each other. This may allow 360 degree rotation of the camera about the rotational axis.

The cables in the embodiments of FIG. 15k further provide an electrically coupling to the camera to carry one or both of electrical power and image data between the probe and the camera. This allows the orienting mechanism to provide both actuation of the camera and data/power transmission without the need for other connections/wireless communication. The cable(s) allows data and/or power to be transmitted via the orienting mechanism so that other cables are not required. The power and data may be transmitted by separate cables forming the orienting mechanism, or using a single cable by modulating the power signal as described elsewhere herein.

FIG. 15l illustrates am embodiment similar that that shown in FIG. 15k in which the self-cleaning module 6 comprises a movable cleaning element 6a mounted on (i.e. fixed relative to) the probe 2 and in contact with the outer surface of the camera 5. The cleaning element is movable relative to the probe body. In this embodiment, the movable cleaning element comprises a rotating element such as a brush. Although illustrated in use with the cable orienting mechanism, the self-cleaning module of FIG. 15l can be used with any embodiment described herein.

FIG. 15m illustrates an embodiment in which power is transmitted wirelessly from the probe to the movable camera 5. FIG. 15m shows a close up of the camera 5 mounted to the distal end of the probe body 9 via the supporting tabs 20a, 20b as already described in connection with other embodiments. In order to wirelessly transfer power the scope comprises one or more probe mounted induction coils (or other types of near field wireless power transmission components) and one or more camera mounted induction coils (or other types of near field wireless power transmission components) arranged to transfer power between each other. The induction coils are arranged to transfer energy by inductive coupling. In the presently described embodiment, a first probe mounted induction coil 302 is provided at a first of the pair of supporting tabs 20a (e.g. at the receptacle or hole at which the pin is received). A corresponding first camera mounted induction coil 304 is provided in the axis pin 20 at the first side 32 of the camera. The reverse may be the case where the pins are on the tabs.

The first probe mounted induction coil 302 is electrically coupled to a cable 18 to provide a source of electrical power. The first camera mounted induction coil 304 is electrically coupled to the camera electronics via a cable 306 within the camera. The probe and camera induction coils are therefore in close proximity to each other such that energy transfer can take place by electromagnetic induction without requiring a wired connection between the two relatively moving parts of the scope. By locating the induction coils at the point of connection between the camera and the probe body the coils remain in close proximity throughout the range of motion of the camera. A similar arrangement of induction coils is provided on the second side 33 of the camera 5. As can be seen in FIG. 15m the second tab 20b of the pair of supporting tabs has a second probe mounted induction coil 308, with a corresponding second camera mounted induction coil 310 provided in the axis pin 20 at the second side 33 of the camera 5. In other embodiments, induction coils may be provided at only one axis pin.

If desired, the scope of the invention can include two cameras 5 to give binocular vision while in other embodiments a single camera 5 uses two lenses to project onto a single sensor to give binocular vision, using out of phase images at full resolution and a filter built into each lens turns opaque at the same frequency to separate the two images.

In another embodiment, two lenses are projected onto a single sensor to give binocular vision, lenslets on the image sensor are tuned to either one lens or the other based on light entry angle, approximately 50% tuned to one lens and approximately 50% to the other and image processing techniques separate the image to give binocular vision, although with reduced resolution.

Referring back to FIGS. 10 to 14, these show another embodiment of the scope 1 of the invention in which the image sensor board 37 is relocated from the eyeball camera 5 as shown in FIGS. 1 to 9 into the probe body 9. The eyeball camera 5 can be rotated as shown in the drawings while the self-cleaning module 6 serves to clean the eyeball camera 5 as previously described.

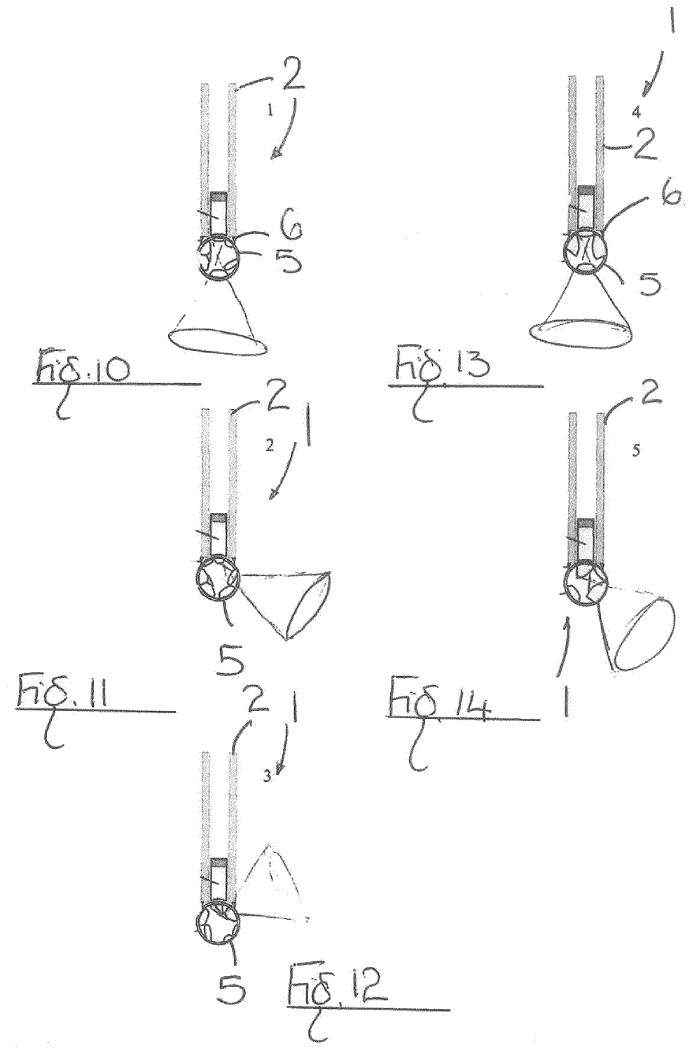

FIGS. 10 to 12 show an eyeball camera 5 in which the image sensor is relocated from the eyeball into the probe which can operate at 0° through the centre, 45° using a lens to one side, and 120° through another side using lenses and prism(s). FIGS. 13 and 14 show another configuration with 0° through the centre and with 45° also through the centre. In one embodiment, optical filters are applied to the lens to prevent light from the wrong lens entering the sensor. If desired, light emitters 42 can be built into the eyeball camera 5 as previously described or, alternatively, the light emitters 42 can be located on the probe 2.

In embodiments where the image sensor is located in the probe, one or more fibre optic cables may be provided to couple light between the lens or lenses and the respective image sensor or sensors. This may allow more flexibility in the relative positioning of the lenses and image sensors.

FIG. 15a shows a schematic representation of a further embodiment of the invention in which the image sensor boards 37 are stacked in the probe 2 to save space and reduce the profile of the probe 2 so that the scope 1 can fit into different lumens in the body. As the image sensors are stacked to save space and the lenses deliver a spatially separated image to each sensor, image processing software can deliver this image in a binocular view to give depth perception with a low-profile probe 2. More particularly, as shown in the drawing, the stacked optical configuration 7 is made up of first and second stacked fixed image sensors 49, 50 and corresponding first and second lens elements 8 directing light to the image sensors 49, 50 together with a slidably adjustable front lens 51. Lens element 49a directs light to sensor 49. Lens element 50a directs light to sensor 50.

In one embodiment aspherical lenses can be used to bypass the first fixed sensor 49 in an efficient manner e.g. crescent profile lenses (in plan view).

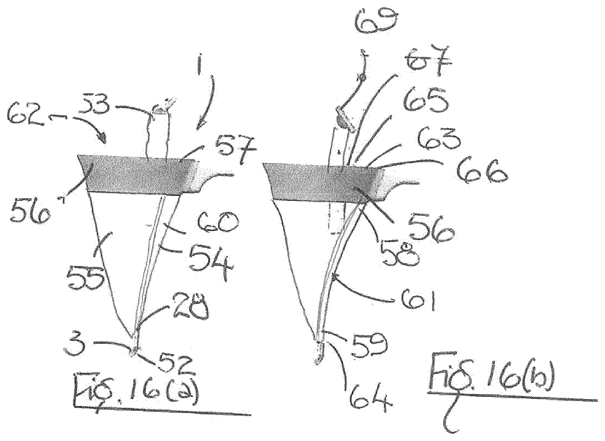

FIG. 16(a) shows a side elevation of a further embodiment of the scope 1 in which the articulatable visualiser 3 is a tiltable camera 52 controllable with an orienting mechanism 4 in the form of a directional lever 53 and the scope 1 is provided with a stabilizer in the form of a speculum 55. More particularly, the speculum 55 is attached to a speculum holder in the form of a speculum handle 56. The speculum handle 56 has a collar 57 and the probe 2 is attached to the collar 57 and located in the speculum 55 with the tiltable camera 52 at the distal end 28 of the probe 2. The probe 2, being integrated with the speculum holder 56 in a unitary structure, defines a unified speculum-probe scope assembly. As previously described, the scope assembly can be in the form of an otoscope for examining ears or a surgical endoscope which allows for bi-manual diagnosis and surgical techniques whilst benefiting from the advantages associated with endoscopic visualisation of the ear.

The speculum 55 is made up of a proximal open end 58 through which a surgeon can access an ear during surgical procedures and a distal insertion end 59 insertable in an ear. A substantially conical speculum wall 60 extends between the proximal and distal ends 58, 59 which defines an internal chamber 61 for receiving the probe 2 and surgical instruments in use. The conical speculum wall 60 further defines a relatively large surgical access opening 62 at a rim 63 at the proximal end 58 and a relatively narrow insertion opening 64 at the distal end 59 through which a surgeon can access the ear during surgical procedures.

The collar 57 of the handle 56 has speculum mounting in the form of a ring 65 defining a bore 66 for receiving the rim 63 of the speculum to mount and secure the handle 56 to the rim 63 of the speculum 55. The collar 57 is further provided with a probe mounting 67 to mount the probe 2 on the handle so that the elongate probe 2 can extend through the speculum from the proximal end 58 to the distal end 59 and exit the distal end 59 through the insertion opening 64 if required.

As shown in the drawing, the orienting or tilting mechanism 4 can be employed to orient the tiltable camera 52 as required. The orienting mechanism 4 is manually controllable with the probe direction lever 53.

FIG. 16(b) is a side elevation of a scope 1 similar to the scope 1 of FIG. 16(a) but in which the probe direction lever is in the form of a manually operable probe knob 69.

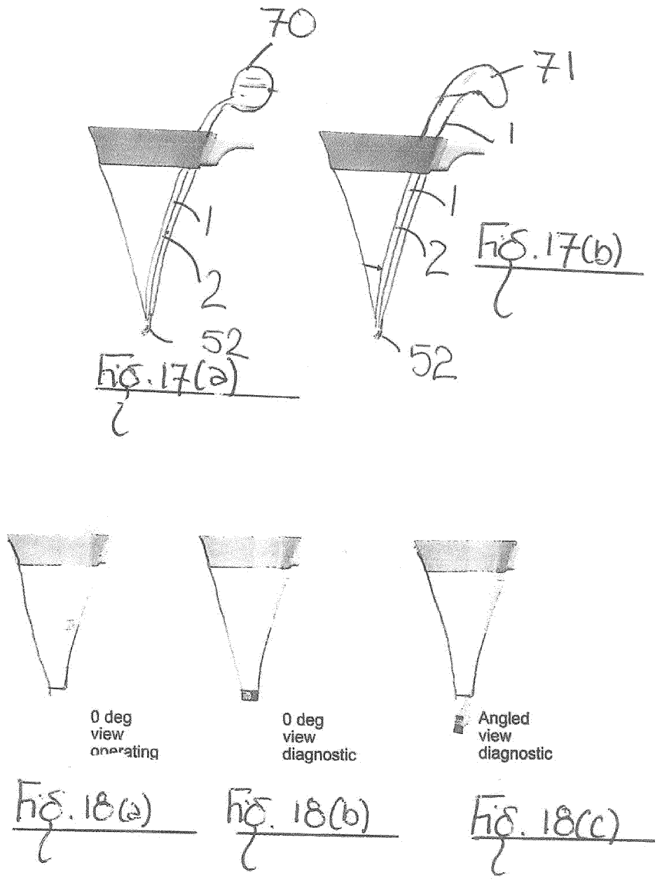

FIG. 17(a) is a side elevation of a fourth embodiment of the scope 1 similar to the scope 1 of FIGS. 16(a) and 16(b) but in which, in addition to the elongate probe 2 being slidable and orientable, the elongate probe 2 is curved to conform with the wall 60 of the speculum 55 to create additional space in the speculum chamber 61 for a surgeon in use. The scope 1 is provided with a probe direction wheel 70 in place of the probe direction lever of FIG. 16(a).

FIG. 17(b) is a side elevation of the endoscope of FIG. 17(a) but in which the probe direction wheel 70 is replaced by a probe direction slider 71.

FIGS. 18(a) to 18(c) show side elevations of an embodiment of the invention similar to that of FIGS. 16 and 17 but in which the probe 2 is also slidable with respect to the speculum 2.

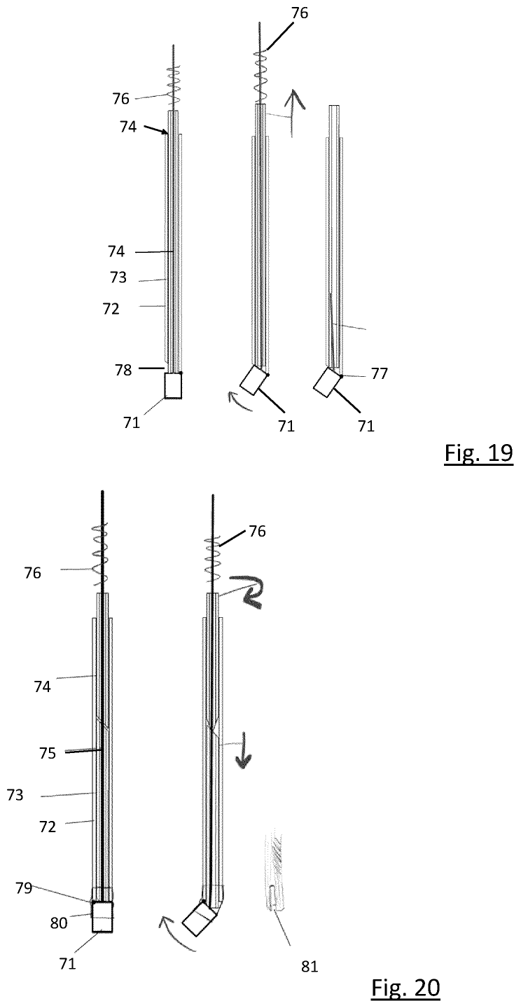

FIG. 19 is an enlarged cross-sectional view of a portion of the orienting mechanism 4 for the tiltable camera 52. As shown in the drawing, the probe orienting mechanism is made up of the tiltable camera 52 including a light source in the form of a camera module 71 at the distal end of the elongate probe 2 which is made up of an outer shaft 72, an inner shaft 73 within the outer shaft 72 and a channel 74 for housing a actuating cable 75 which extends between the camera module 71 and a spring 76 (or other elastic material) of the orienting mechanism 4. The camera module 71 is mounted on the outer shaft 72 at an external living hinge 77 with a tilt recess 78 disposed opposite the external living hinge 77 so that linear movement of the inner shaft 73 relative to the outer shaft 72 causes the camera module 71 to tilt.

The living hinge 77 therefore retains the camera module 71 so that it tilts in a predictable way. In other embodiments of the invention, the living hinge 77 can be replaced by other locators such as a magnet or ball and socket. In still further embodiments, the data cable 18 can double as the actuator cable 75.

The portion of the orienting mechanism 4 of FIG. 19 can also be provided with a protective elastic cover to protect subjects from sharp edges.

FIG. 20 shows an enlarged cross-sectional view of a portion of the probe orienting mechanism 4 similar to the probe orienting mechanism 4 of FIG. 19 (like numerals indicate like parts) but in which the external living hinge 77 is replaced by an internal spring-operated living hinge 79 to facilitates orientation of the camera module 71 by rotary motion of the inner shaft 73 relative to the outer shaft 72. As shown in the drawing, the internal living hinge 79 is provided with a hinge cover 80 and is located at an inner shaft slot 81 so that rotation of the inner shaft 73 causes the camera module 71 to rotate as shown in the drawing.

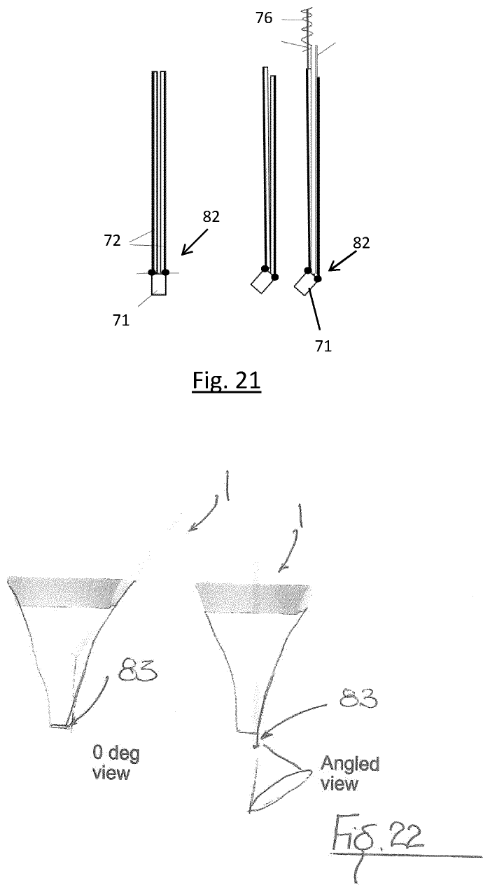

FIG. 21 is an enlarged cross-sectional view of a portion of the orienting mechanism 4 in which a spring-operated double living hinge 82 of the probe facilitates orientation of the camera probe 2. In this embodiment, the outer shaft 72 is fully or partially split allowing the inner and outer sides to move relative to each other.

FIG. 22 shows side elevations of a further embodiment of the scope 1 similar to the scope of FIGS. 16 to 21 but in which the scope 1 is further provided with an angled prism 83 to assist in unobtrusive visualisation.

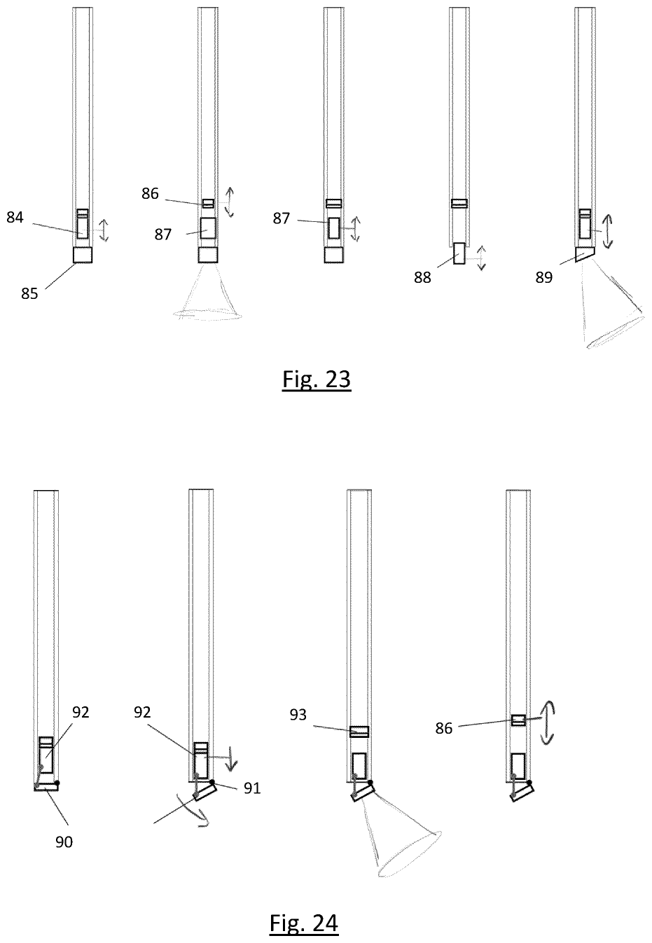

FIG. 23 shows enlarged cross-sectional views of various optical configurations 7 of showing the lens elements 8 of the optical configurations 7 which are movable to focus images. More particularly, the optical configurations 7 can be made up of combinations of an adjustable lens and sensor element 84, a fixed front element 85, and adjustable sensor 86, a rod lens 87, an adjustable front lens 88 and an angled front lens 89.

FIG. 24 shows enlarged cross-sectional views of an alternative optical configuration 7 in which the optical configuration 7 includes a tilting front lens 90 attached to the elongate probe 2 at a lens hinge 91 and mechanically coupled via a mechanical coupling 92 to another element so that as the front lens 90 is tilted, the coupled element moves to ensure image transmission. The optical configuration can be further provided with a fixed sensor 93. Additional moving elements can also be included to adjust the focus such as an adjustable sensor 86.

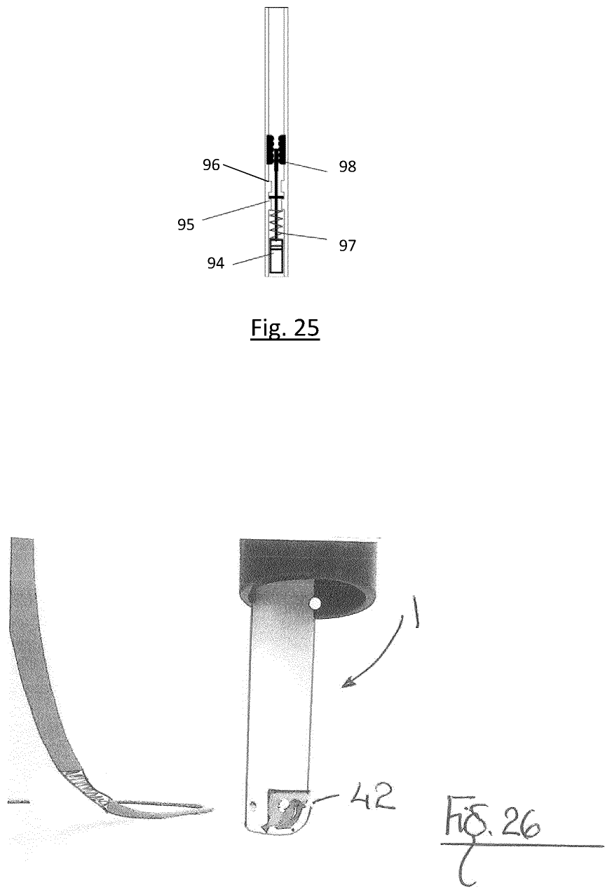

FIG. 25 shows an enlarged cross-sectional view of yet an alternative optical configuration 7 in which the optical configuration 7 is a staged focus optical configuration 7. In the present embodiment, the optical configuration is provided with a lens and/or sensor moving element 94 and a front end stop 95 spaced apart from a rear end stop 96 to determine the position of the lens and sensor moving element. The optical configuration 7 has two focus positions. In one position, the lens and/or sensor moving element 94 is pulled back fully to rest at the first predetermined focus position (the rear end stop 96). In the second position, the lens and/or sensor moving element 94 is pushed fully forward to the second focus position (front end stop 95). The small amount of movement required (potentially under 0.1 mm) to adjust the focus in the required range is achieved with a mechanism consisting of a spring 97 and a solenoid 98. In an alternative embodiment, a spring and actuator under a ratchet mechanism (similar to a click pen) or a friction controlled mechanism can be employed.

The two focus positions firstly allow the surgeon look in detail at the anatomy and secondly allow the camera to be pulled back to view the tools in the surgical field during operations.

FIG. 26 shows a further embodiment of the invention in which the probe 2 is provided with four light emitters 42 surrounding the articulatable visualiser 3.

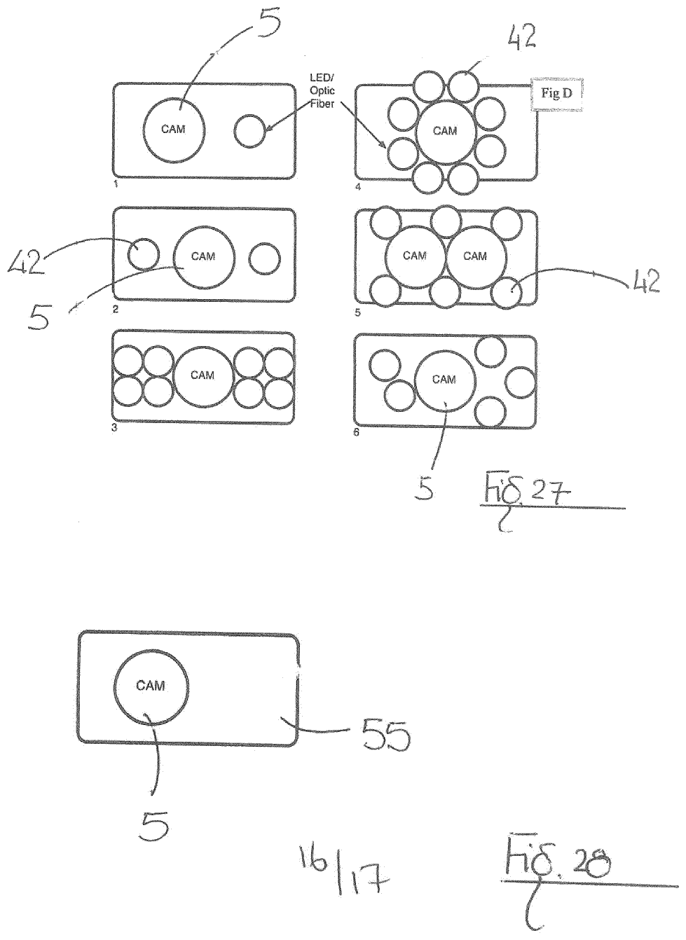

FIG. 27 shows a schematic representation of various light emitter 42 arrangements. Light is delivered through the probe 1 from a light source at the proximal end 27 using for example optic fibers which can be one large optic fiber or multiple optic filters in different formats, including but not limited to those shown in the drawing. In addition, the distal surface of the optic fiber can be planar or curved to allow light dispersion.

As shown in FIG. 28, light can also be transmitted through the structure of the scope 1 e.g. through the speculum 55 to the articulatable visualizer 3.

The scope 1 and systems of the invention can be formed from any suitable materials e.g. biodegradable materials while additive manufacturing is utilized to make the lenses and other components of the copes of the invention.

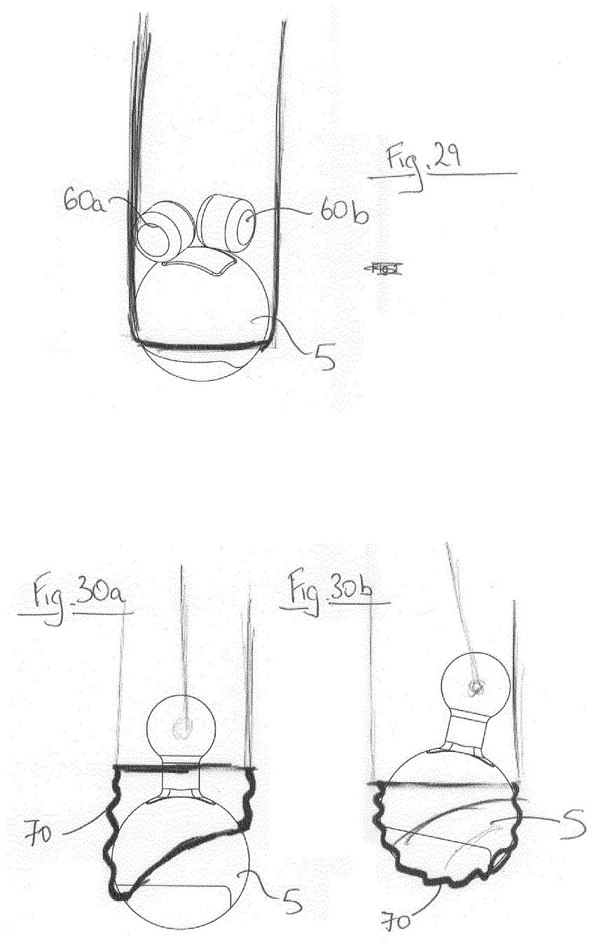

Referring to FIG. 29, an embodiment of the scope of the invention is described in which parts described with reference to previous embodiments are assigned the same reference numerals. In this embodiment, the orientating mechanism for the visualizer 5 comprises a pair of rollers 60a, 60b in contact with the visualizer 5 and configured for rotation about axes which are generally orthogonal to each other. It will be appreciated that the two rollers can be employed in combination to effect rotation of the visualiser about an infinite number of axes.

Referring to FIGS. 30a and 30b, an embodiment of the scope of the invention is described in which parts described with reference to previous embodiments are assigned the same reference numerals. In this embodiment, the self-cleaning module comprises an elastomeric sheath 70 configured for movement distally from a retracted position shown in FIG. 30a to a deployed position shown in FIG. 30b where the sheath closes over the lens of the visualizer 5 wiping it clean.

The following clauses, which are not claims, define optional or preferred features of the present disclosure:

1. A scope for examining or surgically treating an internal structure of the ear comprising:

a probe;

at least one visualiser on the probe having an optical configuration, and

a light source

wherein the visualiser is articulatable relative to the probe by an orienting mechanism for optimal viewing of the internal structure of the ear.

2. A scope as claimed in Clause 1 wherein the visualizer comprises a communication module configured for wirelessly transmitting or receiving data with a communication module in the probe, optionally the probe is configured to wirelessly transmit power to the visualizer.

3. A scope as claimed in Clause 1 or 2 wherein the visualiser is rotatable about a visualiser articulation axis.

4. A scope as claimed in Clause 3 wherein the visualiser has a uniform profile about its articulation axis.

5. A scope as claimed in any preceding Clause wherein the visualiser is substantially spherical and in which the probe optionally comprises a socket for receipt of the substantially spherical visualizer for rotation of the visualiser about multiple axes.

6. A scope as claimed in any of Clauses 1 to 5 wherein the visualiser comprises an image sensor and a lens and is optionally configured to focus the visualizer by adjustment of the distance between the lens and image sensor.

7. A scope as claimed in Clause 6, in which the visualizer is adjustable between two or more pre-set focus settings.

8. A scope as claimed in any of Clauses 1 to 8 wherein the probe exhibits a nonuniform profile, and includes a narrowed profile at a proximal end or a centre part.

9. A scope as claimed in any preceding Clause wherein the orienting mechanism is a belt- or band-driven orienting mechanism fluidic orienting mechanism, or driven using magnetic forces such as a motor, directly acting on the visualiser, or acting on a wheel or linkage in communication with the visualiser.

10. A scope as claimed in any preceding Clause further comprising a self-cleaning module for cleaning detritus from the visualiser.

11. A scope as claimed in Clause 10 wherein the self-cleaning module comprises a cleaning surface and/or a fluid dispenser.

12. A scope as claimed in Clause 11 wherein the self-cleaning module is configured to provide relative movement between the visualizer and the cleaning surface to clean the visualizer.

13. A scope as claimed in any of Clause 1 to 12 further comprising a stabilizer for supporting the scope.

14. A scope as claimed in Clause 13 wherein the stabilizer comprises a speculum.

15. A scope as claimed in any of Clauses 1 to 14 wherein the scope is an endoscope or otoscope.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1-97. (canceled)

98. A scope for examining an internal part of the ear, the scope comprising:
a probe formed by an elongate probe body for insertion into the internal ear structure; and
a camera comprising an image sensor located within a housing;
wherein the housing is rotatably coupled to a distal end of the probe body whereby the camera is rotatable relative to the probe body, and wherein an outer surface of the rotatable camera forms an outer exposed surface of the scope.

99. A scope as claimed in claim 98, wherein:
the image sensor is arranged to visualise through the outer surface of the rotatable camera which forms the outer exposed surface of the scope;
the outer surface of the rotatable camera through which the image sensor is arranged to visualise is spaced apart from the probe body to maintain clearance between them and allow relative movement between them; and
the camera is preferably arranged to rotate 360 degrees about its rotational axis.

100. A scope as claimed in claim 98, wherein:
the probe body comprises one or more tabs supporting the housing extending in a distal direction from the distal end of the probe body; and
the one or more tabs comprise a single tab, the single tab being arranged to support the camera in a cantilever arrangement along the axis of rotation of the camera.

101. A scope as claimed in claim 98, further comprising one or more self-cleaning modules, wherein at least one of the self-cleaning modules comprises a cleaning element and the camera is rotatable relative to the cleaning element.

102. A scope as claimed in claim 101, wherein the cleaning element is positioned proximally or distally relative to the camera.

103. A scope as claimed in claim 101, wherein the cleaning element is fixed in position by attachment to the probe and is arranged to engage with the rotatable camera at any point along the camera circumference as defined by its rotation axis.

104. A scope as claimed in claim 98, wherein:
the camera is fitted with an optical configuration comprising a lens;
the image sensor is configured to generate image data from light received through the lens;
the optical configuration further comprises a focusing mechanism for focusing the lens; and
the focusing mechanism comprises a focusing lens and is arranged to vibrate the focusing lens or the image sensor so that the distance between the focusing lens and the image sensor varies.

105. A scope as claimed in claim 104, wherein the scope, or a data processing system coupled to the scope, is arranged to sample the resulting image data to generate focused images, and the image data is preferably sampled by selecting frames corresponding to a desired focal point along the range of motion of the lens.

106. A scope as claimed in claim 104, wherein:
the vibratable focusing lens or image sensor is arranged to provide cleaning of an outer surface of the camera;
the focusing mechanism is arranged to vibrate the focusing lens or image sensor at a frequency that is approximately the same as the resonant frequency of the lens or window of the camera; and
optionally, the focusing mechanism is arranged to switch the frequency of vibration of the focusing lens or image sensor between a first frequency that is different from the resonant frequency of the lens or window of the camera and a second frequency that is approximately the same as the resonant frequency of the lens or window.

107. A scope as claimed in claim 98, wherein the probe further comprises a light source for illuminating a field of view of the image sensor.

108. A scope as claimed in claim 98, wherein the scope comprises an orienting mechanism configured to control the rotational movement of the housing relative to the probe body.

109. A scope as claimed in claim 108, wherein the orienting mechanism comprises a belt orienting mechanism, preferably comprising a drive belt coupled to a drive shaft positioned at a proximal end of the probe and a driven shaft acting on the housing wherein the drive belt is wrapped around the drive shaft and/or the driven shaft.

110. A scope as claimed in claim 108, wherein:
the orienting mechanism comprises at least one cable coupled to two attachment points on a distal side of the housing, the attachment points being mutually opposed about an axis of rotation of the housing;
the orienting mechanism comprises a first cable arranged to provide rotation of the camera in a first direction and a second cable arranged to provide rotation in a second direction about the rotational axis; and
preferably, the first and second cables are connected to or within the camera and extend in opposite directions about the rotational axis of the camera around the housing of the camera.

111. A scope as claimed in claim 110, wherein the cables enter the camera at the same point on its circumference as defined by the rotation axis, preferably in different planes relative to each other.

112. A scope as claimed in claim 110, wherein the cables are configured to provide rotation in excess of 175 degrees in either direction around the rotational axis of the camera from a center position in which the image sensor points in a distal direction along a longitudinal axis of the probe.

113. A scope as claimed in claim 110, wherein the first and second cables further provide an electrical coupling to the camera to carry one or both of electrical power and image data between the probe and the camera, and at least one cable is connected to the image sensor so as to transmit image data generated by the image sensor, whereby this preferably allows the orienting mechanism to provide both actuation of the camera and data/power transmission without the need for other connections/wireless communication.

114. A scope as claimed in claim 98, wherein the camera has a circumference that is less than 6.5 mm measured through the rotational axis.

115. A method of examining the internal ear structure of a patient, the method comprising:
inserting a probe into the internal ear structure, the probe formed by an elongate probe body supporting a camera comprising an image sensor disposed within a housing, the housing being rotatably coupled to a distal end of the probe body whereby the camera is rotatable relative to the probe body;
rotating the camera relative to the probe body to cause rotational movement of the image sensor relative to the internal ear structure, wherein rotating the housing comprises rotating the housing through at least 90° relative to the probe body whilst viewing the internal ear structure throughout the rotational movement;
and the method further comprises cleaning an outer surface of the camera, wherein:
cleaning the outer surface of the camera comprises rotating the camera relative to a cleaning element, the cleaning element preferably being fixed relative to the probe; and
rotating the camera relative to the cleaning element comprises wiping an outer surface of the housing with the cleaning element.

116. A method of cleaning a scope for examining an internal part of the ear, wherein the scope comprises an image sensor disposed within a rotatable housing and further comprises a cleaning element, the method comprising: rotating the housing relative to the cleaning element; and using the cleaning element to clean an outer surface of the housing; wherein cleaning the outer surface of the housing comprises wiping the housing with the cleaning element by rotating the housing relative to the cleaning element.

117. A method as claimed in claim 116, wherein rotating the housing comprises rotating the housing from a first position in which the image sensor faces in a distal direction to a second position in which the image sensor faces in a proximal direction or to a third position in which the image sensor faces generally perpendicularly relative to a central longitudinal axis of a probe body; and the method further comprises returning the housing to the first position.
